(19) United States
(12) Patent Application Publication
(10) Pub. No.: US 2008/0248097 A1
(43) Pub. Date: Oct. 9, 2008

(54) POLYMERIC MICELLES FOR COMBINATION DRUG DELIVERY

(76) Inventors: Glen S. Kwon, (US); Younsoo Bae, Madison, WI (US); Adam WG. Alani, Madison, WI (US)

Correspondence Address: SCHWEGMAN, LUNDBERG & WOESSNER, P.A., P.O. BOX 2938, MINNEAPOLIS, MN, 55402, US

(21) Appl. No.: 12/037,840

(22) Filed: Feb. 26, 2008

Related U.S. Application Data
(60) Provisional application No. 60/891,632, filed on Feb. 26, 2007.

(51) Int. Cl. A61K 9/127; A61K 47/30; A61K 31/704; A61K 31/335; A61P 35/00; A61K 31/35; A61K 31/337

(52) U.S. Cl. 424/450; 514/772.3; 514/34; 514/183; 514/453; 514/449

(57) ABSTRACT

The invention provides block polymers, micelles, and micelle formulations for combination drug therapy. Polyamide block polymers, such as those of formulas I and II are useful, for example, for preparation of mixed drug micelles, including simply mixed micelles, physically mixed micelles, and chemically mixed micelles. The invention further provides methods of treating cancer, and inhibiting and killing cancer cells. Also provided are methods for the preparation of polymer drug conjugates and intermediates for their synthesis.

### RELATED APPLICATIONS

This application claims priority under 35 U.S.C. §119(e) to U.S. Provisional Patent Application No. 60/891,632, filed Feb. 26, 2007, which is incorporated herein by reference.

### GOVERNMENT SUPPORT

This invention was made with government support under Grant No. AI-043346 from the National Institutes of Health. The United States Government has certain rights in the invention.

### BACKGROUND OF THE INVENTION

The selective augmentation of drug concentrations in avascular tumor tissues is one of the most challenging issues of current cancer chemotherapy using macromolecular bioconjugates. Most anticancer drugs are pharmacologically effective but limited in their clinical applications due to serious toxicity and low water solubility. Improving the biodistribution of these drugs would reduce their overall toxicity and improve the therapeutic effects. For these reasons, interest has centered on the creation of drug carriers that safely and precisely deliver the appropriate amounts of active drugs to solid tumors.

Anticancer drugs are often used in combinations to maximize the efficacy of the cancer chemotherapy while minimizing toxicity. Each anticancer drug, however, has its own pharmacokinetic profile. One drug may interact with another in a way that changes their respective pharmaceutical properties, thereby increasing the risk of side-effects. Accordingly, there is a need for drug delivery methods and systems that can carefully control the amount and release rate of more than one drug. Such a drug delivery system would allow for safe and efficient combination chemotherapy.

Accordingly, there is a need for novel compounds, compositions, and methods for controlled combination chemotherapy. There is also a need for novel compositions, such as micelle bioconjugates, and methods for mediating prodrug delivery to cells. There is a further need for compositions that are stable, have low toxicity toward normal tissue, and can provide for release of therapeutic agents in target tissues or cells.

### SUMMARY

The invention provides pH-responsive micelles that can be used as drug carrier systems. The pH-responsive micelles enable the delivery of a wide range of anticancer drugs to a tumor with precise control of drug type, amount, and release rate. The pH-responsive micelles can incorporate a single type of anticancer agent, or multiple types of drugs, allowing for their simultaneous delivery to the blood stream or to specific body tissues. The diameter of the micelles can be carefully controlled. One class of micelles can have average diameters of less than about 100 nm, with narrow size distribution. This precise control of size and size distribution occurs regardless of drug mixing ratios used in the preparation.

The drug delivery systems can deliver multiple types of drugs to a to the blood stream or to a targeted site in the body, while each type of drug, once released from the micelle, retains its respective pharmacokinetics in a combination context. These drug delivery systems therefore provide chemotherapeutic methods of using a combination of therapeutic agents in a micelle system. The drug combination can produce a synergistic effect so that a lower effective does is required for a suitable therapeutic benefit.

The invention also provides a block polymer comprising a first block and a second block; wherein the first block comprises two or more ethylene glycol segments; the second block comprises two or more amino acid units derived from aspartic acid, glutamic acid, or both; and at least one side chain of an amino acid unit is covalently linked to at least one therapeutic agent through a hydrazide, ester, or amide moiety. The hydrazide, ester, or amide moiety can optionally be linked to the therapeutic agent through a linking group or “linker”, for example, a linker derived from succinic acid, 4-(hydroxymethyl)-benzaldehyde, levulinic acid, an ethanolamine derivative, or a combination thereof.

The amino acids can be D- or L-amino acids. In some embodiments, at least two side chains of the polymer chain are covalently linked to therapeutic agents through hydrazide moieties. In some embodiments, therapeutic agents are linked to the polymer through both hydrazide moieties and ester moieties derived from the side chains of the amino acid segments of the polymer. These therapeutic agents can be the same or different.

When one type of therapeutic agent is attached to a polymer, micelle formulations can be prepared from such polymers in combination with polymers of the invention that have at least one different type of therapeutic agent linked to the polymer, thus providing a physically mixed micelle formulation by combining these two different drug linked polymers into the same micelles.

The hydrazide moiety can be formed by combining the side chain carboxylate moiety of an aspartic acid unit or a glutamic acid unit, a carbonyl moiety of the therapeutic agent or a linker attached to the therapeutic agent (e.g., a ketone or an aldehyde moiety of the agent or linker), and hydrazine or a hydrazine derivative. The therapeutic agent can be linked to the hydrazide moiety at the N′ nitrogen of the hydrazide through a hydrazone bond. The therapeutic agent can be a drug or prodrug, or can be derived from a drug or prodrug.

The two or more ethylene glycol segments can form a poly(ethylene glycol) chain. The chain can be straight, branched, cyclic, or polycyclic. The chain can have a molecular weight of about 400 to about 36,000 g/mol. In one embodiment, ten or more ethylene glycol segments can form a poly(ethylene) glycol chain and the chain can be either straight or branched. The first block can include about 10 to about 600 ethylene glycol segments.

The poly(ethylene glycol) chain can terminate with a hydroxyl group, an alkoxy group, a hydroxyl protecting group, or an optionally substituted amino group. The poly(ethylene glycol) chain can terminate with an amino group substituted by an amino protecting group, such as, for example, an acetate group.

The first block and the second block can be linked to each other through an amide bond or a linking group. The amino acid units of the second block can be derived from D- or L-amino acids. In certain embodiments, the amino acids are L-amino acids. The amino acids of the second block can be, for example, aspartic acid, glutamic acid, a combination thereof, or derivatives thereof. The molecular weight of the second block can be about 350 to about 40,000 g/mol, or about 500 to about 20,000 g/mol. The second block can include about 2 to about 200, about 5 to about 150, about 10 to about 100, or about 10 to about 50 amino acid units.

The polymer can have amino acid side chains that are linked to therapeutic agents. For example, one polymer molecule can have several therapeutic agents attached to it. The therapeutic agents can be the same or different. In one embodiment, there are at least two different types of therapeutic agents linked to each polymer chain. In other embodiments, each polymer chain has therapeutic agents of all the same type. In these embodiments, the polymers can be used to prepare micelles with other types of polymers, e.g., polymers with side chains that have a different type of therapeutic agent on them, thus providing physically mixed polymeric micelles. Not every amino acid side chain need be linked to a therapeutic again. In some embodiments, greater than half of the amino acid side chains of a particular polymer will be linked to therapeutic agents. In other words, the micelles are prepared from numerous polymer chains; some of the side chains of the polymer are linked to a first type of drug, while other side chains are optionally linked to a second type of drug, and some side chains are not linked to a drug.

The therapeutic agents can be anticancer agents, for example, anticancer drugs. The desired therapeutic agents may have low water solubility, thus increasing the need for alternate delivery systems to what is currently available for cancer therapy. Examples of therapeutic agents that can be used to form bioconjugates with the polymers described herein include, but are not limited to, aclarubicin, apicidin, cyclopamine-KAAD, cucurbitacin, dolastatin, doxorubicin (adriamycin), fenritinide, geldanamycin, herbimycin A, 2-methoxyestradiol, paclitaxel, radicicol, rapamycin, triptolide, wortmannin, and the various combinations thereof.

The invention also provides a block polymer comprising a first block and a second block; wherein the first block comprises about 5 to about 600 ethylene glycol segments, or about 10 to about 500 ethylene glycol segments; the second block comprises 5 to about 50 amino acid units derived from aspartic acid, glutamic acid, or both aspartic acid and glutamic acid; and at least one side chain of an amino acid unit is covalently linked to a therapeutic agent through a linker of the formula:

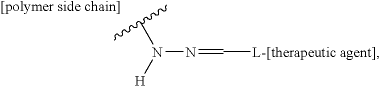

wherein L is a direct bond or a linking group.

The therapeutic agent can be, or can be derived from, any drug, such as a drug that is therapeutically effective for treating cancer, for example, the therapeutic agents listed herein. The hydrazone linkage of the polymers of the invention can be formed from a hydrazide nitrogen and a carbonyl (e.g., an aldehyde or ketone moiety) of an anticancer drug or a linker attached to such drug.

The invention further provides a polymer comprising formula I:

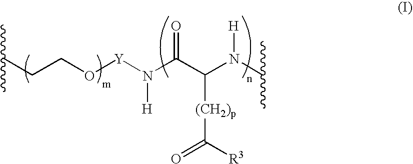

wherein m is about 10 to about 600; n is about 10 to about 100; p is 1, 2, 3, or 4;

Y is a linking group comprising one to twenty carbon atoms, optionally interrupted by one to eight oxygen atoms, nitrogen atoms, or amide groups; and

each R³ is independently OH, a hydroxyl protecting group, —NH—NH₂, or —NH—N═C-L-[drug] where L is a direct bond or a linking group; or a salt thereof.

The group —NH—N═C-L-[drug] can have been formed between a hydrazide group on a side chain of an amino acid moiety of the polymer and a carbonyl group of the drug, or a carbonyl group of a linker attached to a drug. The drugs used in the combination treatment compositions of the invention can be any therapeutically effective drug. Therefore, the group [drug] can be any drug, for example, a drug for treating cancer, such as a heat shock protein 90 inhibitor, for example, an ansamycin, geldanamycin, herbimycin A, radicicol, a synthetic compound that binds to the ATP-binding site of HSP90, and the like. Specific examples of suitable drugs include, but are not limited to, aclarubicin, apicidin, 17-allylamino-17-demethoxygeldanamycin (17-AAG), cyclopamine-KAAD, cucurbitacin, dolastatin, doxorubicin, fenritinide, herbimycin A, geldanamycin, paclitaxel, proteasome inhibitors, radicicol, rapamycin, triptolide, and wortmannin. Any drug that can be covalently bonded to a linking group, which can be then linked to the polymer through a hydrazone bond, can be employed. For example, the drug can be any drug that has a suitably reactive hydroxyl, carboxyl, carbonyl, or amino group that can be attached to the polymer through a linking group. Each R³ group can be the same or several can be different, i.e., the identity of each R³ groups can be determined independent from one another.

The invention yet further provides a polymer of formula II:

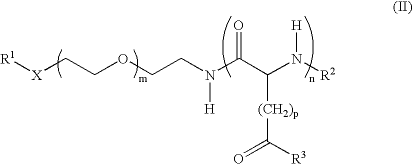

wherein

m is about 10 to about 600; n is about 10 to about 100; p is 1, 2, 3, or 4;

R¹ is H, alkyl, or a hydroxyl or nitrogen protecting group;

X is O, NH, or absent; R² is H or a nitrogen protecting group; and

each R³ is as defined above for formula I;

or a salt thereof. In one embodiment, m can be about 200 to about 300, n can be about 30 to about 50, and p can be 1 or 2.

The invention additionally provides a micelle comprising a plurality of a polymer described above. Therapeutic agents can reside on the inside of the micelle and the ethylene glycol segments of the polymers can align toward the outside surface of the micelle. In one embodiment, more than one type of drug is conjugated to each individual polymer chain of the micelle, i.e., each polymer chain has more than one type of drug linked to it. In another embodiment, only one type of drug is conjugated to each individual polymer chain. In these embodiments, however, these polymer chains are combined with other polymer chains that have a different type of drug linked to them, thus forming mixed polymeric micelles. Under appropriate physiological conditions, the polymers of these mixed polymeric micelles can undergo hydrolysis to provide various combinations and ratios of the different drugs.

The invention also provides a method of inhibiting, or killing, cancer cells that includes contacting the cells with an effective amount of micelles described herein. The micelles described herein can be used to form a pharmaceutical composition by combining them with a pharmaceutically acceptable diluent or carrier.

The invention also provides a method for treating cancer comprising administering to a patient afflicted with cancer a therapeutically effective amount of a pharmaceutical composition that includes the micelles described herein. The cancer treatment can include delivering two or more drugs to a tumor, and wherein the ratio of drug types delivered to the tumor is determined by controlling the ratio of polymers used to prepare the micelles of the pharmaceutical composition. The invention further provides a method of delivering a drug to the blood stream of a mammal comprising intravenously administering a formulation that includes a micelle composition.

The invention also provides a method of delivering a therapeutic agent to an organ or a cell comprising administering a micelle as described herein to the organ or cell, wherein the hydrazone linkers of the micelle polymers side chains hydrolyze to release the therapeutic agents upon encountering a pH of less than about 7. The micelles display pH-dependent drug release as the pH of their environment decreases below 6.0, which corresponds to the condition of intracelluar acidic compartments such as endosomes and lysosomes.

The invention thus provides novel polymers, polymer compositions, including micelles, and methods of making and using the polymers and compositions. For example, the polymers and compositions can be used to treat a disease or disorder of a mammal. Such diseases include cancer, such as the cancers described in U.S. Pat. No. 6,833,373 (McKem et al.). The polymers and compositions can also be used to prepare a medicament to treat a disease in a mammal, for example, cancer in a human. Also provided are useful intermediates for the preparation of the polymers disclosed herein.

### BRIEF DESCRIPTION OF THE DRAWINGS

Embodiments of the invention may be best understood by referring to the following description and accompanying drawings. The description and drawings may highlight a certain specific example, or a certain aspect of the invention, however, one skilled in the art will understand that portions of the highlighted example or aspect may be used in combination with other examples or aspects of the invention, and that certain aspects may be illustrated narrowly for clarity while the scope of the invention is broader than such aspects.

FIG. 1 illustrates polymeric micelles according to an embodiment of the invention: (a) prepared from self-assembling acid-sensitive amphiphilic block copolymers; and (b) in aqueous solution. A supramolecular structure of the micelles has the advantage of site-specific targeting in the mammalian body, while protecting reactive functional moieties with the hydrophilic outer shell during blood circulation.

FIG. 2 illustrates a time and pH-dependent adriamycin (“ADR”) (doxorubicin) release profile of a PEG-p(Asp-Hyd-ADR) micelle, according to an embodiment of the invention. The micelles selectively released ADR under the pH conditions of regions A and B which correspond to outer and intracellular conditions, respectively. The amount of loaded ADR on the micelles was calculated at pH 3.0 in region C, where the release was significantly increased.

FIG. 3 illustrates three different types of mixed micelle formulations of the invention. Polymer drug conjugates with only one type of drug (e.g., drug A) per micelle can be prepared and then combined in a formulation with micelles formed from polymer drug B conjugates to provide a simply mixed micelle formulation for providing combination drug therapy. Micelle formulations can also be prepared from polymers that have more than one type of drug linked to each polymer chain, to form chemically mixed micelles for use in combination drug therapy. Micelles can also be prepared by combining in the same micelles some polymers conjugated to drug A and other polymers conjugated to drug B, thus providing physically mixed micelles for use in combination drug therapy.

FIG. 4 illustrates CLSM images of human small cell lung cancer SBC-3 cells incubated with ADR and PEG-p(Asp-Hyd-ADR) micelles (10 μg mL⁻¹). In contrast to free ADR, the fluorescence of the ADR in the micelles is only detected when they are activated. A series of optical sections was stacked (Z-stacked) by moving the focal plane of the instrument step-by-step through the depth of the cell. The Z-stacked images clearly reveal that the micelles are localized within the cytoplasm with a dot-like shape, assumed to be micelles in acidic lysosomal compartments, while most of the ADR released from the micelles is in the cell nucleus; a) free ADR after 1 hour exposure, b) free ADR after 24 hours incubation, c) micelles after 1 hour exposure, d) micelles after 24 hours incubation.

FIG. 5 illustrates growth inhibition assay results on human small cell lung cancer SBC-3 cells with different ADR concentrations and exposure times. As time elapses, the curve indicating the inhibition effect of the ADR-containing micelles approached that of free ADR.

FIG. 6 illustrates the concept of designing and delivering pH-sensitive polymeric micelles to intracellular acidic regions, according to an embodiment of the invention. Combinations of various drug-conjugate block copolymers can be used to prepare physically mixed or simply mixed micelles for combination drug therapy. By preparing micelles shown in the figure wherein various ADR moieties are replaced with one or more other therapeutic agent-derived moieties, a chemically mixed micelle can be used for the combination drug therapy.

FIG. 7 illustrates biodistribution and tumor specific accumulation of micelles of the invention, and a comparison of plasma levels of doxorubicin and polymer-linked doxorubicin delivered in the micelles described herein, according to an embodiment of the invention. Animal studies confirmed the prolonged circulation in the blood and tumor-specific accumulation of the pH-sensitive micelles.

FIG. 8 illustrates the broader therapeutic window for doxorubicin micelles compared to doxorubicin injection, based on treatment-to-control (T/C) ratio. Cancer treatment efficacy of the pH-sensitive micelles was evaluated by comparing the therapeutic windows of small molecule drugs (doxorubicin) and the doxorubicin-conjugated micelles.

FIG. 9 illustrates the improved effectiveness of combination chemotherapy using mixed micelles as a result of drug accumulation in a cancerous tumor. Initial drug mixing ratio at injection can be preserved within the tumor tissue because the mixed micelles can deliver multiple drugs at the same pharmacokinetic profiles. Combination therapy produces synergism that is greater than the sum of separate treatment regimen.

FIG. 10 illustrates mixed micelles for multiple drug delivery, according to various embodiments of the invention. The schematic illustrates the ‘tunability’ of the polymers of various embodiments, wherein any percentage from about 0.1% to about 99.9% of one drug can be prepared, while the balance of drugs linked to the polymer chain are a different drug conjugate.

FIG. 11 illustrates UV absorbances of various polymer-drug bioconjugates (with varying ratios of doxorubicin and wortmannin on the same polymeric chain) according to various embodiments of the invention.

FIG. 12 illustrates in vitro data for DOX/WOR micelle formulations. The compositions for the mixed polymeric micelles are distinguished with the names ‘chemically mixed micelle (CMM)’ and ‘physically mixed micelle (PMM)’ depending on how mixed micelles were prepared. Cytotoxic activity of combination use of free drugs and mixed polymeric micelles against a human breast cancer MCF-7 cell line at 30 hours (A) and 72 hours (B) after drug exposure. The difference in cellular viability was compared with 50 μM drug concentration (C).

FIG. 13 illustrates examples of DOX/GA mixed micelle formulations. Chemical design and preparation of pH-sensitive polymeric micelles. HSP90 and TOPOII inhibitors have been conjugated to a poly(ethylene glycol)-poly(aspartate-hydrazide) block copolymer through degradable hydrazone linker for pH-responsive drug release control.

FIG. 14 illustrates the viability of MCF-7 breast cancer cells treated with small molecule drugs (A) and micelles (B) through different regimen schedules and combination formulation at normothermia (37° C.). D, G, DM, GM and NT stand for DOX, 17-HEA-GA, DOX-loaded micelle, 17-HEA-GA-loaded micelle, and normothermia, respectively.

FIG. 15 illustrates a comparison of inhibitory concentrations for suppressing 50% cell viability (IC₅₀) for small molecule drugs and polymeric micelles at normothermia (37° C.). D, G, DM and GM stand for DOX, 17-HEA-GA, DOX-loaded micelle and 17-HEA-GA-loaded micelle, respectively.

FIG. 16 illustrates various drugs used to prepare mixed-micelle libraries, according to various embodiments, and the particle size resulting from micelles prepared from their respective polymer conjugates. Data were obtained by dynamic light scattering measurements by using the NICOMP 380 submicron particle analyzer; samples were diluted at 2 mg/mL.

### DETAILED DESCRIPTION

The invention provides polymers, particularly block co-polymers, that can have refined properties making them “tunable” for use in combination drug therapy. Block co-polymers that include poly(ethylene glycol) (“PEG”) segments are of interest because PEG is unique in its ability to facilitate transfer of appended agents across cell membranes. PEG is both water soluble and membrane permeable, therefore its use in the polymers described herein affords several advantages over currently known technology.

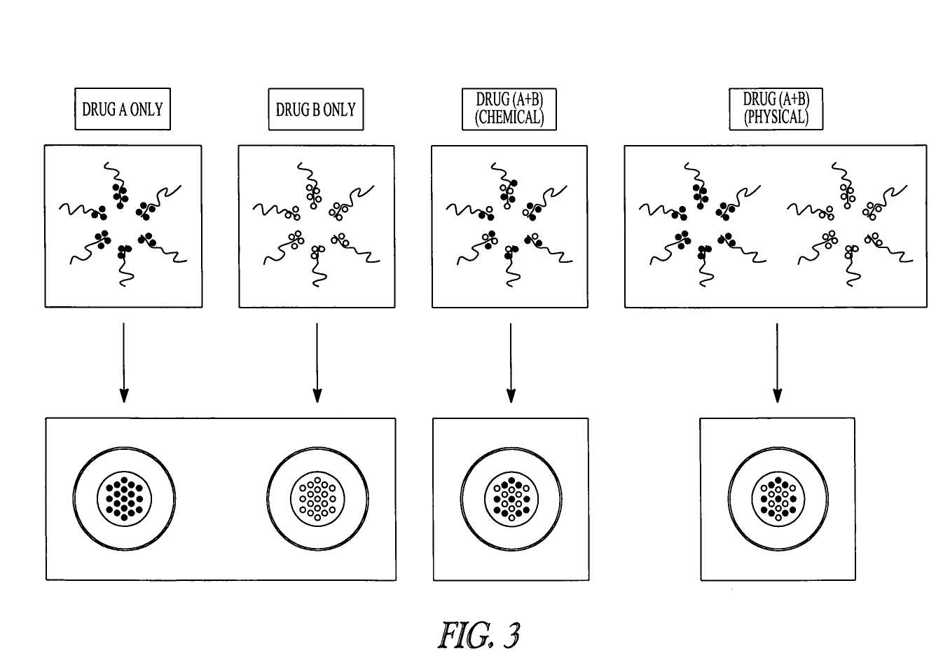

The inclusion of PEG chains onto the polymers disclosed herein allows for the covalent yet labile attachment of therapeutic agents and tunability or modulation of the release of the agents in or around cells. In particular, the appended therapeutic agents of micelle particles can be released upon their hydrolytic removal in response to slightly lower than physiological pH, such as the pH found in cancerous cells, as well as the intracellular compartments such as endosomes and lysosomes. The pH dependence of the drug linked micelles is illustrated FIG. 2.

### DEFINITIONS

References in the specification to “one embodiment”, “an embodiment”, “an example embodiment”, etc., indicate that the embodiment described may include a particular feature, structure, or characteristic, but every embodiment may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

A “block copolymer” refers to a polymer with repeating units of one type adjacent to each other in a linear manner to form a block, with is linked, for example, through a covalent bond to a second block made up of repeating units of a second type, which are adjacent to one another in a linear manner to form a second block of the block copolymer.

The term “therapeutic agent” refers to biologically active agents, prodrugs, or drugs, including, for example, any organic or organometallic small molecule compound (e.g., a molecule with a molecular weight of less than about 500, or less than about 800), polymeric species (including nucleic acids (DNA and RNA), proteins, peptides, hormones, carbohydrates, and derivatives thereof), lipids and mixtures thereof, wherein said drug or agent can be administered in vivo (in humans or animals) for the treatment of a disease, condition, or disorder. Several examples of suitable therapeutic agents can be found in U.S. Pat. No. 6,833,373 (McKem et al.) and the documents cited therein, the disclosure of which is incorporated herein by reference.

Therapeutic agents include signal transduction inhibitors, drugs that may prevent the ability of cancer cells to multiply quickly and invade other tissues. One class of therapeutic agents that can be used in the micelle formulations of the invention include heat shock protein (HSP)90 inhibitors and topoisomerase II inhibitors. HSP90 is a molecular chaperone that forms a complex with topoisomerase II, which is one of its client proteins that play a crucial role in maintaining cell viability. HPS90 inhibitors such as geldanamycin and its analogues (e.g., 17-AAG) bind to N-terminus of HSP90 dimers. Anticancer drugs like doxorubicin target intermediate topoisomerase II complex to induce apoptosis by intercalating into DNA. The therapeutic agents described herein can provide synergistic therapeutic effects when included in the micelle formulations of the invention.

Specific examples of therapeutic agents of the invention that can be used to form bioconjugates with the polymers described herein include, but are not limited to, aclarubicin, apicidin, 17-allylamino-17-demethoxygeldanamycin (17-AAG), cyclopamine-KAAD, cucurbitacin, docetaxel, dolastatin, doxorubicin (adriamycin), geldanamycin, fenritinide, herbimycin A, 2-methoxyestradiol (an angiogenesis inhibitor), paclitaxel, radicicol, rapamycin, triptolide, wortmannin, and the various combinations thereof. Other therapeutic agents include proteasome inhibitors such as bortezomib, and benzyloxycarbonyl-L-leucyl-L-leucyl-L-leucinal (“Z-Leu-Leu-Leu-H (aldehyde)”), which is also a potent inhibitor of Cathepsin K. See Votta et al., J. Bone Miner. Res., 12, 1396 (1997). Additional therapeutic agents that have suitably reactive carbonyl groups, or groups that can employ a linker, that can be used to form bioconjugates can be found in The Merck Index, 12^{th} Edition (1996).

Further specific examples of suitable therapeutic agents that can be linked to the polymers to prepare micelle formulations of the invention include aclacinomycins, 9-aminocamptothecin, aminopterin, ara-C (cytarabine), azaserine, biricodar, bleomycins, cactinomycin, calusterone, camptothecin, carboplatin, carboquone, caminomycin, carubicin, chlormadinone acetate, chromomycins, cisplatin, CPT 11, cyclophosphamide, cytarbin, cytosine arabinoside, dactinomycin, daunorubicin, 6-diazo-5-oxo-L-norleucine, dichloromethotrexate, docetaxel, doxorubicin, dromostanolone propionate, dromostanolone, emitefur, epirubicin, estramustine, etoposide, exemestane, flavopiridol, 5-fluorouracil, formestane, gemcitabine, hexamethyl melamine, idarubicin, irinotecan, leurosidine, medroxyprogesterone, megestrol acetate, melengestrol, melphalan, menogaril, 6-mercaptopurine, methopterin, methotrexate, methoxsalen, mitomycin-C, mitoxantrone, nogalamycin, onapristone, phenesterine, pipobroman, piposulfan, pirarubicin, podophyllotoxin, porfiromycin, prednimustine, rubitecan, sobuzoxane, spironolactone, streptonigrin, teniposide, tenuazonic acid, testolactone, topotecan, tretinoin, triaziquone, trimetrexate, uredepa, valrubicin, valspodar, vinblastine, vincamine, vincristine, vindesine, and zorubicin. Each of these drugs has at least one hydroxyl, carboxyl, ketone, or amine group that can form a bond with a linker of the invention for use in therapeutic micelles of the invention.

Other specific therapeutic agents that can be employed in the micelle formulations of the invention, optionally by covalently bonding the agent the a polymer with a linker, include antineoplastic agents such as tipifamib, gefitinib, cetuximab, oxaliplatin, ansamitocin, arabinosyl adenine, mercaptopolylysine, busulfan, chlorambucil, mitotane, procarbazine hydrochloride, plicamycin, aminoglutethimide, estramustine phosphate sodium, flutamide, leuprolide acetate, tamoxifen citrate, trilostane, amsacrine, asparaginase, interferon, vinblastine sulfate, vincristine sulfate, carzelesin, taxotane, daunomycin; anti-inflammatory agents such as indomethacin, ibuprofen, ketoprofen, dichlofenac, piroxicam, tenoxicam, naproxen, aspirin, and acetaminophen; sex hormones such as testosterone, estrogen, progestone, estradiol; antihypertensive agents such as captopril, ramipril, terazosin, minoxidil, and parazosin; antiemetics such as ondansetron and granisetron; antibiotics such as metronidazole, and fusidic acid; cyclosporine; prostaglandins; biphenyl dimethyl dicarboxylic acid, antifungal agents such as ketoconazole, and amphotericin B; steroids such as triamcinolone acetonide, hydrocortisone, dexamethasone, prednisolone, and betamethasone; cyclosporine, and functionally equivalent analogues, derivatives, or combinations thereof.

As used herein, the drug names adriamycin and doxorubicin are used interchangeably in the context of forming a drug conjugate. The term “adriamycin” is sometimes used to specifically refer to the HCl salt of doxorubicin. Therefore, one skilled in the art would readily recognize that both doxorubicin and its HCl salt will form the same drug conjugate, in various embodiments of the invention.

The term “therapeutically effective amount” is intended to qualify the amount of a therapeutic agent required to relieve to some extent one or more of the symptoms of a disease or disorder, including, but not limited to: 1) reduction in the number of cancer cells; 2) reduction in tumor size; 3) inhibition of (i.e., slowing to some extent, preferably stopping) cancer cell infiltration into peripheral organs; 3) inhibition of (i.e., slowing to some extent, preferably stopping) tumor metastasis; 4) inhibition, to some extent, of tumor growth; 5) relieving or reducing to some extent one or more of the symptoms associated with the disorder; and/or 6) relieving or reducing the side effects associated with the administration of anticancer agents.

The terms “treat” and “treatment” refer to any process, action, application, therapy, or the like, wherein a mammal, including a human being, is subject to medical aid with the object of improving the mammal's condition, directly or indirectly.

The term “inhibition,” in the context of neoplasia, tumor growth or tumor cell growth, may be assessed by delayed appearance of primary or secondary tumors, slowed development of primary or secondary tumors, decreased occurrence of primary or secondary tumors, slowed or decreased severity of secondary effects of disease, arrested tumor growth and regression of tumors, among others. In the extreme, complete inhibition, can be referred to as prevention or chemoprevention.

The term “micelle” refers to a supermolecular structure having a core-shell form. Micelle formation is entropy driven and water molecules are typically excluded into the bulk phase. When above the critical micelle concentration (CMC), amphiphilic portions of the polymer employed aggregate into structured micelles. Polymeric micelles are typically spherical and can have nanoscopic dimensions in the range of about 1 to about 250 nm, typically in the 20-100 nm range. This is advantageous because circulating particles less than about 200 nm can avoid filtering by interendothelial cell slits at the spleen. Polymeric micelles have been shown to circulate in the blood for prolonged periods and capable of targeted delivery of therapeutic agents, for example, nucleic acids or poorly water-soluble compounds. Upon disassociation, micelle unimers are typically <50,000 g/mol, permitting elimination by the kidneys. These properties allow for prolonged circulation with little or no buildup of micelle components in the liver that could lead to storage diseases.

As used herein, the phrases “mixed-micelle” or “mixed-drug micelle” generally refer to any micelle composition or formulation that includes more than one kind of drug attached to the polymers of the micelles. A micelle formulation refers to a group of micelles in a suitable carrier, such as a solution suitable for administration to a human. Three different types of micelle formulations are provided by the invention: simply different micelle formulations, physically mixed micelle formulations, and chemically mixed micelle formulations. Each of these formulations results in micelles containing more than one type of therapeutic agent, thereby providing for combination therapy that can provide synergistic therapeutic effects. Three different types of mixed micelle formulations are illustrated schematically in FIG. 3.

A “simply different” micelle formulation refers to a formulation that has two different types of micelles, wherein a first polymer of the invention is linked to a first drug type to form one type of micelle, and a second polymer of the invention is linked to a second drug type to form a second type of micelle. The two types of micelles are then mixed together in a preparation to form a simply different micelle formulation.

A “physically mixed” micelle formulation includes substantially one type of micelle, prepared from different types of polymers of the invention (different by virtue of the type of drug linked to it), where a first polymer of the invention is linked to a first drug type, and separately, other first polymers of the invention are linked to a second drug type, and the different polymers are mixed together in the same micelle self-assembly process to form substantially one type of micelle, a physically mixed micelle formulation. A physically mixed polymer is thus prepared from polymer chains, each having only one kind of drug linked to them, and more than one different type of polymer chain is used to prepare the micelle.

A “chemically mixed” micelle formulation includes substantially one type of micelle, prepared from one type of polymer of the invention, where both a first drug type and a second drug type are linked to the same polymer chain. These polymers having more than one type of drug linked to them are then formed into micelles to form substantially one type of micelle, a chemically mixed micelle formulation. Therefore, a chemically mixed micelle is prepared from polymers that have more than one kind of drug linked to each individual polymer that forms each micelle.

The term “PEG” refers to poly(ethylene glycol) and derivatives thereof. The molecular weight of the PEG chain can be about 500 to about 20,000. In certain embodiments, the PEG group can have a molecular weight of about 2,000 to about 15,000, about 3,500 to about 12,000, or about 3,000 to about 9,000. In other embodiments, the PEG groups can have a molecular weight of about 4,000 or about 7,000. PEG derivatives include PEG groups with amine or amide groups at one or both ends, and carboxylic acid groups at one or both ends.

The term “linker” or “linking group” refers to a covalent bond or a chain, typically a carbon chain, for example, a C₁-C₂₀ chain, that covalently links two moieties together. The chain is optionally interrupted by one or more nitrogen atoms, oxygen atoms, carbonyl groups, (substituted)aromatic rings, or peptide bonds, and/or one of these groups may occur at one or both ends of the chain that forms the linker. Therefore, either or both ends of the linker can terminate in an oxy, amino, carboxyl, oxycarbonyl, amide, carbonate, carbamate, sulfonyl, or hydrazone group. Accordingly, the linker can also be a chain of one to about five amino acids, of the same type, such as poly L-glycine, poly L-glutamine, or poly L-lysine, or of different types of amino acids. In some embodiments, the linker can be a PEG group, with up to 20 repeating units. Examples of simple linkers include succinimidyl groups, sulfosuccinimidyl groups, maleimidyl groups, and various C₂-C₁₂ diamines and dicarboxylic acids. Many linkers are well known in the art, and can be used to link a polymer described herein to another therapeutic agent. See for example, the linkers described by Sewald and Jakubke in Peptides: Chemistry and Biology, Wiley-VCH, Weinheim (2002), pages 212-223; and by Dorwald in Organic Synthesis on Solid Phase, Wiley-VCH, Weinheim (2002).

The term “protecting group” refers to any group which, when bound to a hydroxyl, nitrogen, or other heteroatom prevents undesired reactions from occurring at this group and which can be removed by conventional chemical or enzymatic steps to reestablish the ‘unprotected’ hydroxyl, nitrogen, or other heteroatom group. The particular removable group employed is often interchangeable with other groups in various synthetic routes. Certain removable protecting groups include conventional substituents such as, for example, allyl, benzyl, acetyl, chloroacetyl, thiobenzyl, benzylidine, phenacyl, methyl methoxy, silyl ethers (e.g., trimethylsilyl (TMS), t-butyl-diphenylsilyl (TBDPS), or t-butyldimethylsilyl (TBS)) and any other group that can be introduced chemically onto a hydroxyl functionality and later selectively removed either by chemical or enzymatic methods in mild conditions compatible with the nature of the product.

A large number of protecting groups and corresponding chemical cleavage reactions are described in Protective Groups in Organic Synthesis, Theodora W. Greene (John Wiley & Sons, Inc., New York, 1991, ISBN 0-471-62301-6) (“Greene”, which is incorporated herein by reference in its entirety). Greene describes many nitrogen protecting groups, for example, amide-forming groups. In particular, see Chapter 1, Protecting Groups An Overview, pages 1-20, Chapter 2, Hydroxyl Protecting Groups, pages 21-94, Chapter 4, Carboxyl Protecting Groups, pages 118-154, and Chapter 5, Carbonyl Protecting Groups, pages 155-184. See also Kocienski, Philip J.; Protecting Groups (Georg Thieme Verlag Stuttgart, New York, 1994), which is incorporated herein by reference in its entirety. Some specific protecting groups that can be employed in conjunction with the methods of the invention are discussed below.

Typical nitrogen protecting groups described in Greene (pages 14-118) include benzyl ethers, silyl ethers, esters including sulfonic acid esters, carbonates, sulfates, and sulfonates. For example, suitable nitrogen protecting groups include substituted methyl ethers; substituted ethyl ethers; p-chlorophenyl, p-methoxyphenyl, 2,4-dinitrophenyl, benzyl; substituted benzyl ethers (p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2- and 4-picolyl, diphenylmethyl, 5-dibenzosuberyl, triphenylmethyl, p-methoxyphenyl-diphenylmethyl, di(p-methoxyphenyl)phenylmethyl, tri(p-methoxyphenyl)methyl, 1,3-benzodithiolan-2-yl, benzisothiazolyl S,S-dioxido); silyl ethers (silyloxy groups) (trimethylsilyl, triethylsilyl, triisopropylsilyl, dimethylisopropylsilyl, diethylisopropylsilyl, dimethylthexylsilyl, t-butyldimethylsilyl, t-butyldiphenylsilyl, tribenzylsilyl, tri-p-xylylsilyl, triphenylsilyl, diphenylmethylsilyl, t-butylmethoxy-phenylsilyl); esters (formate, benzoylformate, acetate, choroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), pivaloate, adamantoate, crotonate, 4-methoxycrotonate, benzoate, p-phenylbenzoate, 2,4,6-trimethylbenzoate (mesitoate)); carbonates (methyl, 9-fluorenylmethyl, ethyl, 2,2,2-trichloroethyl, 2-(trimethylsilyl)ethyl, 2-(phenylsulfonyl)ethyl, 2-(triphenylphosphonio)ethyl, isobutyl, vinyl, allyl, p-nitrophenyl, benzyl, p-methoxybenzyl, 3,4-dimethoxybenzyl, o-nitrobenzyl, p-nitrobenzyl, S-benzyl thiocarbonate, 4-ethoxy-1-naphthyl, methyl dithiocarbonate); groups with assisted cleavage (2-iodobenzoate, 4-azidobutyrate, 4-nitro-4-methylpentanoate, o-(dibromomethyl)benzoate, 2-formylbenzenesulfonate, 2-(methylthiomethoxy)ethyl carbonate, 4-(methylthiomethoxy)butyrate, miscellaneous esters (2,6-dichloro-4-methylphenoxyacetate, 2,6-dichloro-4-(1,1,3,3 tetramethylbutyl)phenoxyacetate, 2,4-bis(1,1-dimethylpropyl)phenoxyacetate, chlorodiphenylacetate, isobutyrate, monosuccinate, (E)-2-methyl-2-butenoate (tigloate), o-(methoxycarbonyl)benzoate, p-poly-benzoate, α-naphthoate, nitrate, alkyl N,N,N′,N′-tetramethyl-phosphorodiamidate, n-phenylcarbamate, borate, 2,4-dinitrophenylsulfenate); and sulfonates (sulfate, methanesulfonate (mesylate), benzylsulfonate, tosylate, triflate).

The phrase “pharmaceutically acceptable” refers to those compounds, materials, compositions, and/or dosage forms that are, within the scope of sound medical judgment, suitable for use in contact with the tissues of human beings and animals without excessive toxicity, irritation, allergic response, or other problems or complications commensurate with a reasonable benefit/risk ratio.

The phrase “pharmaceutically acceptable salts” refers to ionic compounds wherein a parent non-ionic compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include conventional non-toxic salts and quaternary ammonium salts of the parent compound formed, for example, from non-toxic inorganic or organic acids. Non-toxic salts can include those derived from inorganic acids such as hydrochloric, hydrobromic, hydroiodic, sulfuric, sulfamic, phosphoric, nitric and the like. Salts prepared from organic acids can include those such as acetic, 2-acetoxybenzoic, ascorbic, benzenesulfonic, behenic, benzoic, citric, ethanesulfonic, ethane disulfonic, formic, fumaric, gentisinic, glucaronic, gluconic, glutamic, glycolic, hydroxymaleic, isethionic, isonicotinic, lactic, maleic, malic, methanesulfonic, oxalic, pamoic (1,1′-methylene-bis-(2-hydroxy-3-naphthoate)), pantothenic, phenylacetic, propionic, salicylic, sulfanilic, toluenesulfonic, stearic, succinic, tartaric, bitartaric, and the like. Certain compounds can form pharmaceutically acceptable salts with various amino acids. For a review on pharmaceutically acceptable salts see Berge et al., J. Pharm. Sci. 1977, 66(1), 1-19, which is incorporated herein by reference. In certain embodiments, it may be useful to employ salts of various organic moieties on the polymers of the invention. For example, the polyamide block polymer may include one or more acidic or basic side chains that may form salts under appropriate conditions.

The pharmaceutically acceptable salts of the compounds described herein can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., (1985), 1418, the disclosure of which is incorporated herein by reference.

The phrase “low water solubility” refers to a compound that dissolves in water in an amount of less than about 200 μg/mL, for example, measured at neutral pH. In some embodiments, compounds that have low water solubility will dissolve at less than about 100 μg/mL. In other embodiments, low water solubility refers to solubility of less than about 75 μg/mL, less than about 50 μg/mL, or less than about 25 μg/mL. Many drugs are lipophilic, and therefore have poor water solubility, making it difficult to administer them in a safe and effective manner. Suitable water solubility is of particular importance for parenteral administration, therefore the micelle formulations described herein provide a significant advantage for administering these drugs, particularly for administering drugs in combination therapy.

### Variations of Certain Aspects of the Polymers

In a polymer of the invention, two or more ethylene glycol segments can form a poly(ethylene glycol) (“PEG”) chain. Typically this number will be much greater than two segments, such as about 5, about 10, about 20, about 50, about 100, about 200, about 300, about 400, about 500, about 600, or about 800 segments, or any range in between any two of the aforementioned values. The chain can have a molecular weight of about 200 to about 40,000 g/mol. Some embodiments will have PEG moieties of about 300 to about 30,000 g/mol, or about 400 to about 20,000 g/mol. Certain embodiments can have PEG moieties with molecular weights of about 5,000, about 6,000, about 8,000, about 10,000, about 12,000, about 15,000, about 20,000, about 25,000, or about 30,000, or any range in between any two of the aforementioned values. These PEG groups can be single chains, double chains, branched chains, or cyclic or polycyclic groups. In certain circumstances, higher molecular weight PEG chains may be useful to increase the solubility of block copolymers in conjugating multiple types of water-insoluble drugs and/or molecules.

The amino acid units of the second block can be derived from L-amino acids, or alternatively, D-amino acids, or combinations of L- and D-amino acids. The molecular weight of the second block can be about 500 to about 20,000 g/mol. The second block can include about 10 to about 100 amino acid units. Certain specific embodiments can have any number of amino acid between about 10 and 100 units, for example, about 20-60 amino acid units, about 30-50 amino acid units, or about 35-45 amino acid units. In one embodiment, the polymer forming the micelles have a PEG chain of about 12,000 g/mol and an amino acid-derived block of about 35-40 amino acids. This combination provides highly stable micelles.

The mixed micelles can produce similar or identical pharmacokinetics for all of the incorporated drugs, a property not exhibited by any known multiple drug delivery system. In addition, the concentrations and ratios of drugs loaded in the micelles are controllable. The relative proportion of the various incorporated drugs can be optimized to produce the desired synergistic activity from the drug combination. Efficient and safe combination chemotherapy can be achieved using mixed micelle systems by their targeted delivery of optimal ratios of drug molecules.

### Preparation of Polyamide Drug Conjugates

Polyamide segments can be prepared by methods known to those of skill in the art. Other methods, such as those provided in the Examples below, provide for the efficient synthesis of various polyamides useful for preparing the micelles of the invention. The amino acid side chains of the polyamides can then be modified, for example, by removing protecting groups, attaching hydrazide groups, attaching drug conjugates, linkers, and the like. Various linkers can be employed to prepare polyamides with side groups that degrade under certain physiological conditions. For example, hydrazone linkers can be used to link drugs to a polyamide backbone. Hydrazone linkers provide the advantage of molecular stability at neutral pH, while allowing for the hydrolytic cleavage of the hydrazones to release the drugs in an acidic environment, such as the higher acidity typically found in the vasculature of tumors.

Additionally, the linkers can be used to link polymer side chains to therapeutic agents that do not have suitably reactive carbonyl groups to condense with the hydrazine moiety of the polymer side chain. For example, a therapeutic agent of interest that does not possess a reactive carbonyl group may have a suitably reactive hydroxyl or carboxyl group that can be used to form an ester or amide with a linking group. Although these functional groups may hydrolyze (or be cleaved by an enzyme) at slower rates than the hydrazone bonds of the standard linking groups of the invention, these slower cleavage rates can be advantageously used to design delayed release formulations. The release rate of the formulations can be tuned by adjusting the amount of hydrazide linkages and, for example, ester linkages, to the drugs of a micelle polymer, in order to provide a desired release rate of one drug compared to the release rate of another.

For example, geldanamycin can be readily substituted at its C17 position with a variety of alkyl amine nucleophiles. The alkyl chain can then serve as a linking group to the block copolymers of the invention. The linking groups can include carbonyl groups on their chain that will condense with the hydrazide moieties of the polyamide block side chains. An examples of a suitable linker for geldanamycin is 2-aminoacetaldehyde, or a carbonyl group-protected derivative thereof. As illustrated in the scheme below, the 2-aminoacetaldehyde cleanly displaces the C17-methoxy group of geldanamycin to provide “geldanamycin-CHO”.

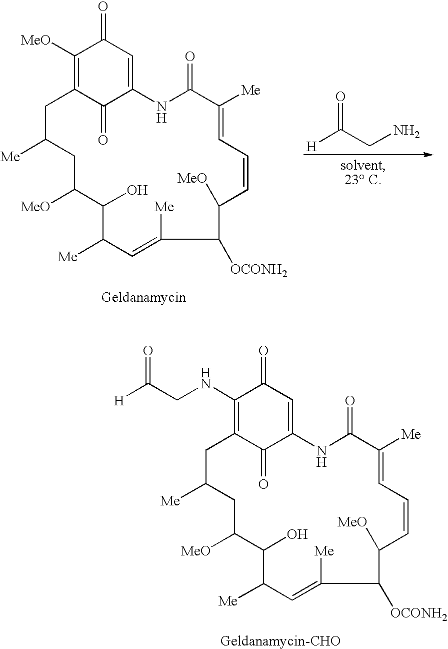

The reaction proceeds smoothly at room temperature in a suitable solvent, such as chloroform. Completion of the reaction is clearly indicated by a significant color change of the reaction mixture. The acetaldehyde moiety serves as an excellent linking group because the aldehyde moiety readily condenses with the hydrazone moiety of a polyamide side chain. This drug linked polymer can then be used to prepare drug delivery micelle formulations.

Table 1 below shows several geldanamycin derivatives that can be prepared using analogous reactions. The derivatives having linkers with suitably reactive carbonyls can also be linked to the polyamides disclosed herein.

TABLE 1

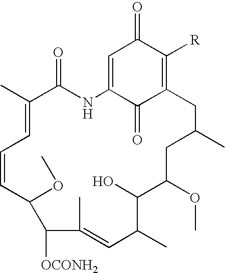

| GA Derivative | Linking Group |
|---|---|
| 1. GA | 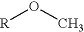 |
| 2. 17-AAG | 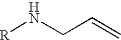 |
| 3. GA(OH) | 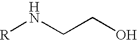 |
| 4. GA(CHO) | 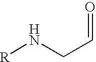 |
| 5. GA(COO-Lev) | 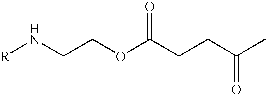 |
| 6. GA(Hyd) | 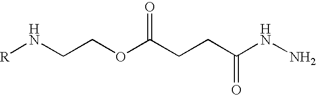 |
| 7. GA(COO-M4) | 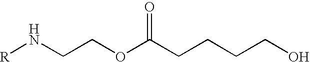 |
| 8. GA(COO-Ali-CHO) | 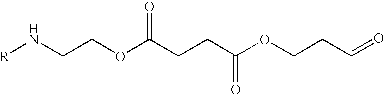 |
| 9. GA(COO-Aro-CHO) | 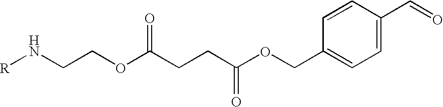 |

These and similar linkers can be used with other therapeutic agents that do not themselves possess carbonyl groups that can condense with the hydrazide moieties of the relevant polyamides. Certain linkers may need additional synthetic manipulations for desired purposes, however these transformations can typically be carried out by one skilled in the art. For example, terminal hydroxyl groups can be converted into leaving groups, such as mesylates and triflates. The leaving groups can then react with various polyamide side chain moieties, such as carboxylic acids, to form ester linkages. In this manner, drug-linked polymers can be prepared with more than one type of linkage, for example, both hydrazone and ester linkages to therapeutic agents. These polymers can then be used to prepare the micelles of the invention, wherein the agents will have different release rates.

### Micelle Preparation

Micelles can be prepared by various methods, including cosolvent evaporation methods. Micelles can typically be prepared making solutions of the polymers disclosed herein. For example, a polymer can be dissolved in a water miscible solvent system. The solution can be slowly added to a vigorously stirred aqueous solution, followed by solvent evaporation or dialysis. The resulting composition can be nanofiltered and/or centrifuged to remove unwanted material. Other useful techniques for preparing micelles have been reported by Kwon and coworkers, Pharm. Res. 2004, 21, 1184-1191.

One useful aspect of micelle carriers is that they can be employed for the delivery of therapeutic agents without chemically modifying the agent. The structure of the polymers described herein can be tailored in order to enhance the properties of the micelles for therapeutic agent delivery. Such tailoring includes varying the amount and nature of amino acid side chain modifications, such as those described in the Examples below.

Micelles formed from the polymers disclosed herein allow for the PEG groups of the polymers to concentrate at outer portions of the micelles. The micelle corona is therefore hydrophilic and allows for prolonged circulation in blood and eventually its incorporation into cells.

One advantage of micelle compositions includes their ease of storage and delivery. Micelle compositions can be lyophilized and reconstituted before intravenous administration. This allows for a lower risk of agent precipitation, which can in some cases lead to embolism formation. Micelle compositions are capable of long blood circulation, low mononuclear phagocyte uptake, and low levels of renal excretion. Also, micelle compositions have enhanced permeability and retention (EPR) to increase the likelihood of their encapsulated therapeutics reaching their targets, for example, tumors.

Tumors typically have high vascular density, as well as defective vasculature. Accordingly, high extravasation occurs and there may be impaired lymphatic clearance. The endocytosis and subsequent micelle disagrregation allows for the release of the encapsulated agent its delivery into the cell.

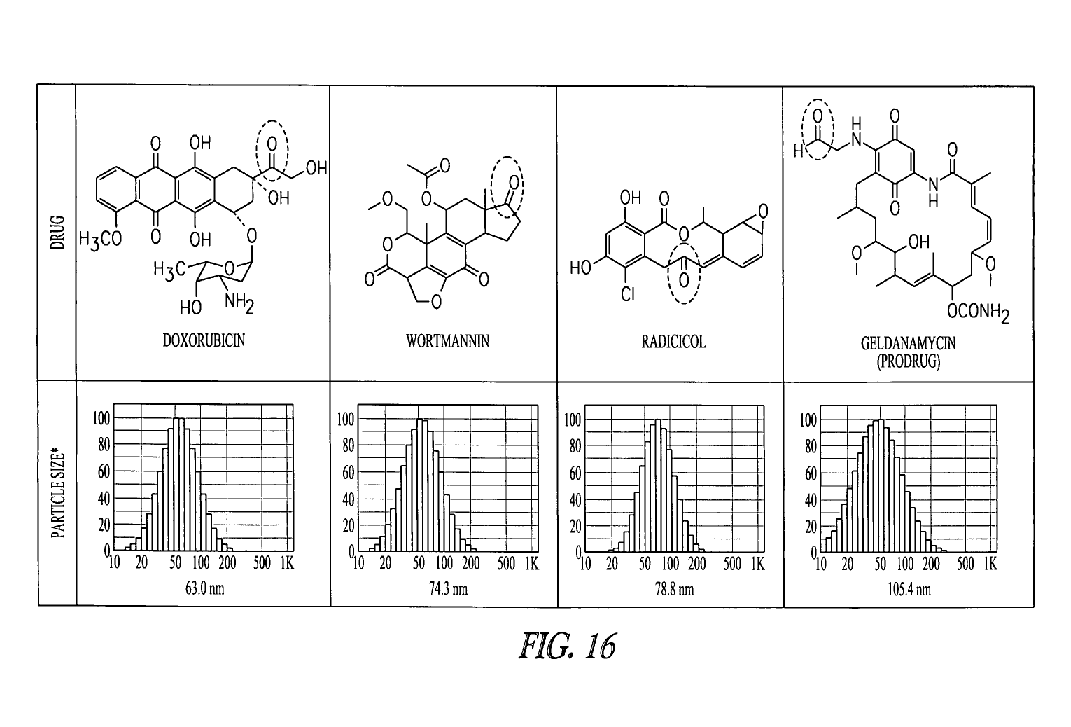

Micelles of various diameters can be prepared, including polyplex micelles. In various embodiments, the unloaded or empty micelles can be prepared. In other embodiments, the resultant micelles can have average diameters of less than about 200 nm, or less than about 100 nm. In another embodiment, the micelles can have an average diameter of between about 55 nm and about 90 nm. In one embodiment, cumulant diameters of micelles can be about 60 nm to about 90 nm. Data for the particle sizes of several drug conjugated polymers that have been prepared is shown in FIG. 16.

The small size of polymeric micelles that have PEG coronas can help the micelle carrier to stay unrecognized, as self, in a biological system. Other advantages associated with nanoscopic dimensions of polymeric micelles include the ease of sterilization via filtration and safety of administration. The core of the micelles can take up, protect and retain biologically active agents, leading to improved solubility and stability of the agents in vivo, their controlled release, and overall reduced toxicity and attenuated pharmacokinetic interaction with other treatment agents.

Related micelles and their uses are described by Kanayama, Kataoka, and coworkers, Chem. Med. Chem. 2006, 1, 439-444, which is incorporated herein by reference. Other related technology is disclosed by Fukushima, Kataoka, and coworkers, J. Am. Chem. Soc. 2005, 127, 2810-2811, which is incorporated herein by reference. Additionally, photochemical transfection technology is disclosed by Kataoka and coworkers, J. Controlled Release 2006, 115, 208-215, which is also incorporated herein by reference. Other useful information on polyamides and micelle technology can be found in WO 2005/118672 (Lavasanifar and Kwon), and U.S. Patent Application Publication Nos. 2004/0005351 (Kwon et al.), 2004/0116360 (Kwon et al.), 2006/0251710 (Kwon et al.), each of which is incorporated herein by reference.

### Micelle Administration

Micelles can be suitably formulated into pharmaceutical compositions for administration to human subjects in a biologically compatible form suitable for administration in vivo. Accordingly, in certain embodiments, a pharmaceutical composition is provided that includes micelles as described herein, in admixture with a suitable diluent or carrier. Suitable diluents or carriers include saline or aqueous dextrose, for example, a 5% aqueous dextrose solution. Such formulations can be prepared so that they are isotonic with human fluids, such as blood, or various tissue environments. In certain embodiments, it may also be desirable to prepare hypertonic or hypotonic preparations. In other embodiments, the composition can be prepared and used for in vitro experimentation, for example, in various screens and diagnostic procedures.

The compositions containing micelles can be prepared by known methods for the preparation of pharmaceutically acceptable compositions that can be administered to subjects, such that an effective quantity of the therapeutic agent within the micelles is combined in a mixture with a pharmaceutically acceptable vehicle. Suitable vehicles are described, for example, in Remington's Pharmaceutical Sciences (2003, 20^{th} Ed.), in The United States Pharmacopeia: The National Formulary (USP 24 NF19) published in 1999, and in the Handbook of Pharmaceutical Additives (compiled by Michael and Irene Ash, Gower Publishing Limited, Aldershot, England (1995)). On this basis, the compositions include, albeit not exclusively, solutions of the micelles in association with one or more pharmaceutically acceptable vehicles or diluents, and contained in buffered solutions with a suitable pH and iso-osmotic with the physiological fluids. In this regard, reference can be made to U.S. Pat. No. 5,843,456 (Paoletti et al.). In one embodiment, the pharmaceutical compositions can be used to enhance biodistribution and drug delivery of therapeutic agents, such as a drug linked to a polymer of the micelle.

The micelles described herein can be administered to a subject in a variety of forms depending on the route of administration selected, as is readily understood by those of skill in the art. The micelles can be administered, for example, by oral, parenteral, buccal, sublingual, nasal, rectal, patch, pump, or transdermal administration and the pharmaceutical compositions formulated accordingly. Parenteral administration includes intravenous, intraperitoneal, subcutaneous, intramuscular, intrasternal, transepithelial, nasal, intrapulmonary, intrathecal, rectal and infusion modes of administration. Parenteral administration may be by continuous infusion over a selected period of time.

Injectable preparations, for example, sterile injectable aqueous or oleaginous suspensions can be formulated according to the known art using suitable dispersing or wetting agents and suspending agents. The sterile injectable preparation can also be a sterile injectable solution or suspension in a nontoxic parenterally acceptable diluent or solvent, for example, as a solution in 1,3-butanediol. Among the acceptable vehicles and solvents that can be employed are water, Ringer's solution, and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid find use in the preparation of injectables. Dimethyl acetamide, surfactants including ionic and non-ionic detergents, polyethylene glycols can be used. Mixtures of solvents and wetting agents can also be useful.

A micelle may be orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, the micelle of the invention may be incorporated with excipient and used in the form of ingestible tablets, buccal tablets, troches, capsules, elixirs, suspensions, syrups, wafers, and the like. A micelle may also be administered parenterally.

Solutions of a micelle can be prepared in water suitably mixed with suitable excipients. Under ordinary conditions of storage and use, these preparations may contain a preservative, for example, to prevent the growth of microorganisms.

The pharmaceutical forms suitable for injectable use include sterile aqueous solutions or dispersion and sterile powders for the extemporaneous preparation of sterile injectable solutions or dispersions. The formulation should be sterile and should be fluid to the extent that the solution or dispersion can be administered via syringe.

Compositions for nasal administration may conveniently be formulated as aerosols, drops, gels and powders. Aerosol formulations typically comprise a solution or fine suspension of the active substance in a physiologically acceptable aqueous or non-aqueous solvent and are usually presented in single or multidose quantities in sterile form in a sealed container, which can take the form of a cartridge or refill for use with an atomizing device. Alternatively, the sealed container may be a unitary dispensing device such as a single dose nasal inhaler or an aerosol dispenser fitted with a metering valve which is intended for disposal after use. Where the dosage form comprises an aerosol dispenser, it will contain a propellant which can be a compressed gas such as compressed air or an organic propellant such as fluorochlorohydrocarbon. The aerosol dosage forms can also take the form of a pump-atomizer.

Compositions suitable for buccal or sublingual administration include tablets, lozenges, and pastilles, wherein the active ingredient is formulated with a carrier such as sugar, acacia, tragacanth, or gelatin′ and glycerine. Compositions for rectal administration are conveniently in the form of suppositories containing a conventional suppository base such as cocoa butter.

The compositions described herein can be administered to an animal alone or in combination with pharmaceutically acceptable carriers, as noted above, the proportion of which is determined by the solubility and chemical nature of the compound, chosen route of administration and standard pharmaceutical practice. In an embodiment, the pharmaceutical compositions are administered in a convenient manner such as by direct application to the infected site, e.g. by injection (subcutaneous, intravenous, parenteral, etc.). In case of respiratory infections, it may be desirable to administer the micelles of the invention and compositions comprising same, through known techniques in the art, for example by inhalation. Depending on the route of administration (e.g. injection, oral, or inhalation, etc.), the pharmaceutical compositions or micelles or biologically active agents in the micelles of the invention may be coated in a material to protect the micelles or agents from the action of enzymes, acids, and other natural conditions that may inactivate certain properties of the composition or its encapsulated agent.

In addition to pharmaceutical compositions, compositions for non-pharmaceutical purposes are also included within the scope of the invention. Such non-pharmaceutical purposes may include the preparation of cosmetic formulations, or for the preparation of diagnostic or research tools. In one embodiment, the therapeutic agents or micelles comprising such agents can be labeled with labels known in the art, such as florescent or radio-labels, or the like. In some embodiments, one or more of the drugs of the polymer can be replaces with a diagnostic agent.

The invention also provides a delivery system that can be used to deliver biologically active agents or formulations or pharmaceutical compositions. In one embodiment, the invention includes the delivery of a combination of cancer therapeutic agents. In another embodiment, the invention includes delivery of therapeutic agents by linking the agents to polymers that self-assemble into micelles comprising a amphiphilic or hydrophobic core and a hydrophilic outer surface, thus improving their delivery in aqueous mediums, such as blood, body fluids, tissues, and organs.

In other aspects, the invention includes the delivery of biologically active agents while reducing their toxicity profile. This is often effectuated by the synergy derived from the administration of a combination of therapeutic agents, thereby reducing the dose required for an equivalent therapeutic effect. The invention also includes a method for reducing aggregation or precipitation of drugs in delivery vehicles, a common problem associated with currently used vehicles for drug solubilization and delivery. As such, the invention provides improved biodistribution of therapeutic agents, resulting in decreased toxicity and/or improved therapeutic efficacy at lower doses. For example, the combined dose used in the combination therapy of the invention can be used to deliver a larger amount of drugs than could be provided as a single dose of one drug, without concomitant toxicity issues that would be encountered if that larger dose was provided by the single drug.

Another aspect of the invention includes a method of delivering biologically active agents to treat a disease, condition, or disorder in a subject in need thereof comprising administering an effect amount of an agent-loaded micelle to a subject. In one embodiment, the disease, condition or disorder is cancer or drug resistant cancers, infectious disease or an autoimmune disease.

The dosage of the micelles of the invention can vary depending on many factors such as the pharmacodynamic properties of the micelle, the biologically active agent, the rate of release of the agent from the micelles, the mode of administration, the age, health and weight of the recipient, the nature and extent of the symptoms, the frequency of the treatment and the type of concurrent treatment, if any, and the clearance rate of the agent and/or micelle in the subject to be treated.

For example, in some embodiments, a dose of a micelle formulation equivalent to about 1 mg mL⁻¹ to about 100 mg mL⁻¹ can be administered to a patient. In certain other embodiments, the micelle formulation includes about 2-20, about 5-15, or about 10 mg mL⁻¹. The specific doses of the compounds administered according to this invention to obtain therapeutic and/or prophylactic effects will, of course, be determined by the particular circumstances surrounding the case, including, for example, the compounds administered, the route of administration, the condition being treated and the individual being treated. A typical daily dose (administered in single or in divided doses) can contain a dosage level of from about 0.01 mg/kg to about 150 mg/kg of body weight of an active therapeutic agent described herein. In some embodiments, about 5-10, about 10-20, about 20-40, about 25-50, about 50-75, about 75-100, or about 100-150 150 mg/kg of body weight of a therapeutic agent are provided in a dose. In other embodiments, about 5, 10, 15, 20, 25, 30, 40, 50, 60, 70, 75, 80, 90, 100, 110, 120, 125, 140, or 150 mg/kg of body weight of a therapeutic agent are delivered in a dose. Often times, daily doses generally will be from about 0.05 mg/kg to about 20 mg/kg and ideally from about 0.1 mg/kg to about 10 mg/kg.

One of skilled in the art can determine the appropriate dosage based on the above factors. The micelles may be administered initially in a suitable dosage that may be adjusted as required, depending on the clinical response. For ex vivo treatment of cells over a short period, for example for 30 minutes to 1 hour or longer, higher doses of micelles may be used than for long term in vivo therapy.

The micelles can be used alone or in combination with other agents that treat the same and/or another condition, disease or disorder. In another embodiment, where either or both the micelle or biologically active agent is labeled, one can conduct in vivo or in vitro studies for determining optimal dose ranges, drug loading concentrations and size of micelles and targeted drug delivery for a variety of diseases.

### Combination Therapy

The polymers, micelles, and micelle formulations of the invention provide advantageous methods for the delivery of a combination of poorly water-soluble therapeutic agents, which are frequently incompatible in commonly encountered delivery vehicles. Because the drugs linked to the polymers only hydrolyze in an acidic environment, the delivery of the therapeutic agent is very controlled. The micelles can accommodate high levels of drug loading while maintaining low toxicity because the drugs are not released at an appreciable rate when not in the vicinity of a tumor. In addition to their tumor specific accumulation, the micelles also offer long circulation in the blood, and the regeneration of active drugs from prodrugs at the targeted site. Furthermore, the use of pH-responsive polymeric micelles reduces non-specific drug distribution, thereby enhancing both the safety of the anticancer drugs and the efficiency of the tumor-targeted delivery, all while delivering two or more drugs simultaneously.

The delivery of two or more therapeutic agents is commonly known as combination therapy. The phrase “combination therapy” (or “co-therapy”) embraces the administration of two different therapeutic agents as part of a specific treatment regimen intended to provide a beneficial effect from the co-action of these therapeutic agents. The beneficial effect of the combination includes, but is not limited to, pharmacokinetic or pharmacodynamic co-action resulting from the combination of therapeutic agents. Administration of these therapeutic agents in combination typically is carried out over a defined time period (usually minutes, hours, days or weeks depending upon the combination selected).

Combination drug therapy typically has inherent difficulties with suitable administration because most drugs are highly water insoluble. Accordingly, oral and intravenous administration can be problematic and ineffective. A significant advantage of the combination therapy that can be administered using the micelles of the invention is that two or more otherwise difficult-to-administer agents, such as low solubility agents, can be in a simultaneous manner. Simultaneous administration can be accomplished, for example, by administering to the subject a single micelle formulation having a fixed ratio of each therapeutic agent. Simultaneous administration of the combination of therapeutic agents can be effected by any appropriate route including, but not limited to, oral routes, intravenous routes, intramuscular routes, and direct absorption through mucous membrane tissues. Separate co-therapies can be administered by the same route or by different routes.

“Combination therapy” also can embrace the administration of the therapeutic agents as described above in further combination with other biologically active ingredients (such as, but not limited to, a third and different therapeutic agent) and non-drug therapies (such as, but not limited to, surgery or radiation treatment). Where the combination therapy further comprises radiation treatment, the radiation treatment may be conducted at any suitable time so long as a beneficial effect from the co-action of the combination of the therapeutic agents and radiation treatment is achieved. For example, in appropriate cases, the beneficial effect is still achieved when the radiation treatment is temporally removed from the administration of the therapeutic agents, perhaps by days or even weeks.

The phrases “low dose” or “low dose amount”, in characterizing a therapeutically effective amount of the therapeutic agents in the combination therapy, defines a quantity of such agent, or a range of quantity of such agent, that is capable of improving the disorder or disease severity while reducing or avoiding one or more therapeutic-agent-induced side effects, such as myelosupression, cardiac toxicity, alopecia, nausea or vomiting.

Many synergistic drug combinations can be administered using the micelle compositions of the invention. One synergistic combination of significant importance is a micelle formulation that includes 17-AAG and paclitaxel. The synergy of 17-AAG and paclitaxel is discussed by Rosen and coworkers (Cancer Research 63, 2139-2144, May 1, 2003; which is incorporated by reference). In one embodiment of the invention, one drug of the micelle formulation sensitizes tumor cells apoptosis induced by the second drug. These synergistic effects can be especially valuable for treating breast cancer.

It is particularly advantageous to deliver combinations of therapeutic agents in a ratio that is non-antagonistic, and especially that is non-antagonistic over a wide range of concentrations. As described in PCT publication PCT/CA02/01500, algorithms are available such that, based on the results of in vitro tests, non-antagonistic ratios may be determined. Examples of suitable synergistic drug combinations and further discussion of determining non-antagonistic ratios over a wide range of drug concentrations can be found in WO 2006/014626 (Mayer et al.), which is incorporated herein by reference.

The following Examples are intended to illustrate the above invention and should not be construed as to narrow its scope. One skilled in the art will readily recognize that the Examples suggest many other ways in which the present invention could be practiced. It should be understood that many variations and modifications may be made while remaining within the scope of the invention.

### EXAMPLES

### Example 1

### Preparation of Mixed Drug Micelles for Combination Chemotherapy

### Introduction

Controlled drug delivery systems containing multiple drugs can avoid unwanted changes in pharmacokinetic profiles and increased risk of side effects by achieving efficient and safe methods for combination chemotherapy. In this Example, a “chemically mixed micelle” is described that can deliver doxorubicin (DOX; a widely used anthracycline) and wortmannin (WOR; a phosphatidylinositol 3-kinase inhibitor) to tumor tissues simultaneously. DOX and WOR were conjugated to α-methoxy-poly(ethylene glycol)-poly(aspartate hydrazide)⁽¹⁻²) through acid-sensitive linkers in various mixing ratios. For the α-methoxy-poly(ethylene glycol)-poly(aspartate hydrazide) preparation, see Y. Bae et al., Bioconjugate Chem. 16: 122-30 (2005); and Y. Bae et al., Angew. Chem. Int. Ed. 42: 4640-3 (2003). The micelles were designed to selectively release the drugs in the cell interior by reacting to a pH decrease in the endosomes and lysosomes (e.g., at a pH of less than about 6.0). This Example illustrates how the precise control of drug loading, drug solubilization of two drugs, and an investigation of synergistic effects induced by the mixed micelles can provide an improved drug delivery system and methods for cancer cytotoxicity.

### Materials and Methods

### Preparation of β-benzyl-L-aspartate N-carboxy-anhydride (“BLA-NCA”)

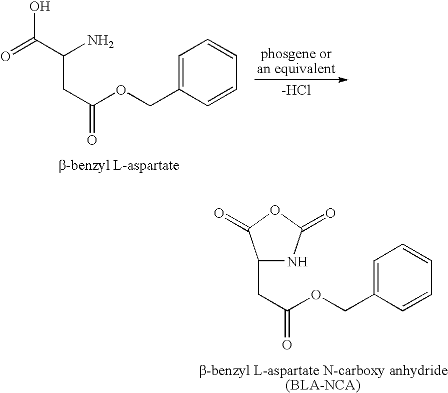

Triphosgene (5.77 g) was added to 0-benzyl L-aspartate (10 g) in dry THF (150 mL), and the reaction was allowed to proceed at 40° C. until the solution became clear. β-Benzyl L-aspartate N-carboxy-anhydride (BLA-NCA) was purified by recrystallization from hexane.

### Synthesis of α-methoxy-poly(ethylene glycol)-poly(β-benzyl L-aspartate) Block Copolymers (“PEG-PBLA”)

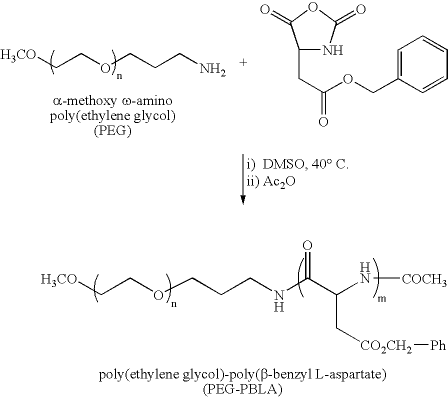

BLA-NCA (1.7 g) was polymerized in DMSO (7 mL) at 40° C. for 2 days by using α-methoxy-ω)-amino-poly(ethylene glycol) (PEG-NH₂; MW=12,000, 2 g) as a macro initiator to obtain methoxy-poly(ethylene glycol)-poly(β-benzyl L-aspartate) (“PEG-PBLA”). The ω-amino group of PEG-PBLA was protected by acetic anhydride after completion of the polymerization reaction.

### Modification of the Side Chains of PEG-PBLA to Provide Drug Binding Hydrazide Linkers (“PEG-p(Hyd)”)

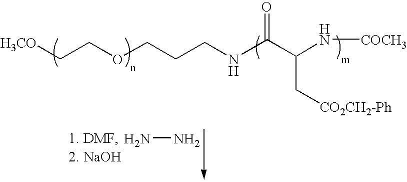

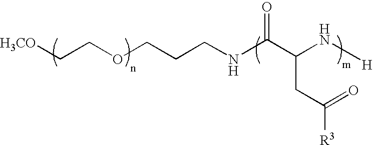

PEG-poly(aspartate hydrazide) [PEG-p(Asp-Hyd)] for R³ = —NH—NH₂

| | | |
|---|---|---|
| R³ = | —NH—NH₂ | ~80% |
| | —OBn | 0-4% |
| | —OH | ~20% |

About 80% of the side-chain benzyl esters of PEG-PBLA were substituted with hydrazide groups for drug binding. PEG-PBLA (500 mg) was dissolved in DMF (10 mL). Anhydrous hydrazine (0.8 equiv. with respect to benzyl groups) was then added to the polymer solution under an Argon atmosphere. The reaction was allowed to proceed at 40° C. for 1 hour, followed by deprotection of remained benzyl groups with 0.1 N NaOH aqueous solution at 25° C. for 1 hour. The polymers were dialyzed against 0.25% ammonia solution and freeze-dried to provide the product, methoxy-poly(ethylene glycol)-poly(aspartate hydrazide) [PEG-p(Hyd)].

### Drug Conjugation and Preparation of Chemically Mixed Drug Micelles

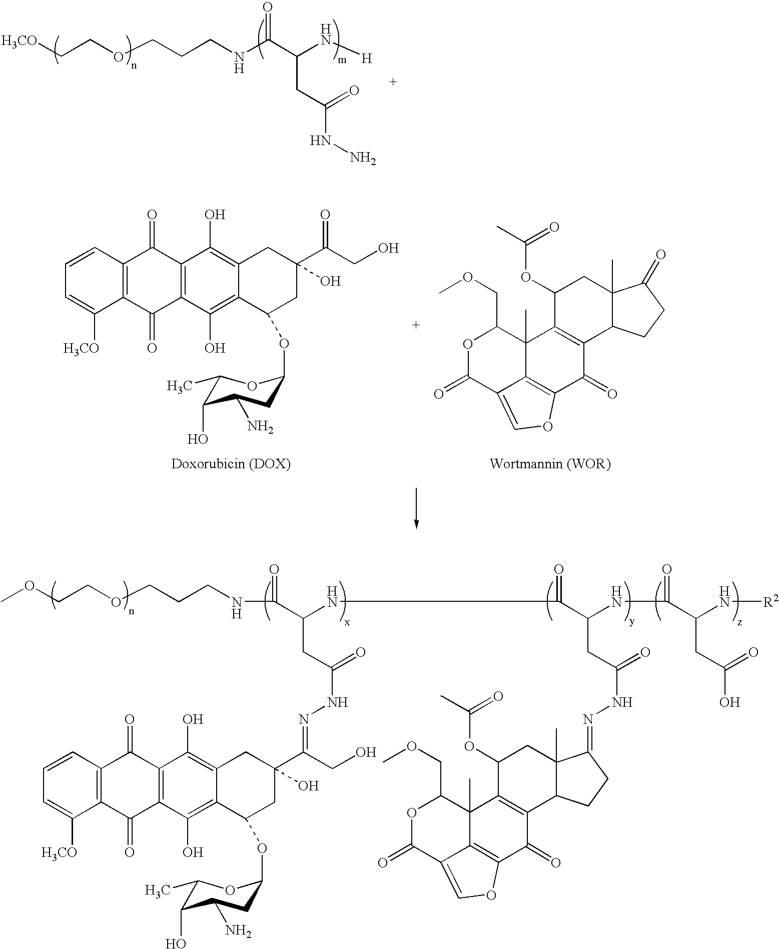

DOX and WOR were conjugated to the hydrazide groups of PEG-p(Hyd) through an acid-sensitive hydrazone linkage at their 13-C and 17-C positions, respectively. PEG-p(Hyd) (100 mg) was dissolved in DMSO (20 mL). The solution was mixed with 2 equivalents of drugs with respect to the number of hydrazide groups of the polymers.

Drug mixture ratios of DOX and WOR used in various trials were 100:0, 75:25, 50:50, 25:75 and 0:100. The mixed solutions were stirred at 25° C. for 3 days. Unreacted extra drugs were removed by precipitation from ether, followed by gel filtration using Sephadex LH20. The polymers were collected by freeze-drying. Drug-conjugated polymers were redissolved in DMSO (5 mg/mL) and diluted 1000 times with Tris-HCl buffered solution (pH 7.4). DMSO was removed from the solution by centrifugal ultrafiltration. Drug loading contents were determined by UV, and the concentrated micelles solutions were stored in 4° C.

### Results

GPC and ¹H-NMR measurements of the PEG-PBLA have revealed that the molecular weight was 19,769, the polydispersity index was 1.18, and the degree of polymerization was 40. It was determined that typically about 31 hydrazide groups (77.5%) were introduced to the side chain of PEG-PBLA to produce the PEG-p(Asp-Hyd). UV measurements demonstrated that DOX and WOR were efficiently conjugated to the polymers with relatively high drug loading contents (25-29 wt. %). Most notably, drug loading ratios for each drug-polymer conjugates were controllable. The micelles prepared from these polymers showed narrow distribution with a P.D.I <0.2, and the average particle sizes were about 100 nm, which is optimal for in vivo drug delivery. See N. Nishiyama, et al., Drug Discov. Today: Technologies 2: 21-6 (2005); and A.

Lavasanifar at al., Adv. Drug. Deliv. Rev. 54: 169-90 (2002).

### Conclusion

Mixed drug micelles that can incorporate multiple anticancer drugs, DOX and WOR, were successfully prepared, providing a single carrier system that simultaneously carried both drugs. These micelles were designed to selectively release drugs by reacting to pH levels in the body, for example, in tumors. The type of drug loading and the drug ratios in the micelles are also controllable by making appropriate modifications of the micelle preparation. Thus efficient and safe combination chemotherapy can be achieved by using the micelle formulations described herein.

Additionally, cytotoxicity results for the combination of doxorubicin and wortmannin have been obtained. Based on pH-sensitive doxorubicin polymeric micelle results, this drug combination is believed to show an additive or synergistic anti-tumor efficacy in a murine tumor model. A further advantage of the micelle formulations is that drug combinations involving drugs with different mechanisms of actions can often be dosed higher than the total dose of a single drug, while lowering the occurrence of side effects because the drugs are not released substantially until the micelle carriers accumulate in tumors.

### Example 2

### Intracellular Drug Delivery by Polymeric Micelles Responsive to Intracellular pH Change

The recent development of biomolecular devices that function within the living body has required the integration of capabilities for sensing in vivo chemical stimuli, generating detectable signals, and effecting suitable responses into a single molecule or molecular complex. In particular, biopharmaceutical systems which interact with intracellular components or events such as ions, proteins, enzymes, and pH changes are becoming important for implementing programmed functions that respond to signatures of the body. Supramolecular chemistry is attracting attention as it offers methods for assembling different constituents capable of structural and dynamic changes into single molecules. Herein we demonstrate the intracellular localization of a pH sensitive supramolecular assembly that changes its structure and fluoresces when activated to induce mortality of malignant cells.

There are many difficulties in the clinical use of some biomolecular devices, these problems include phagocytic clearance during blood circulation, systemic spread causing toxic side effects, and exclusion from the cell by membrane transporters. In general, the cells selectively permeable membranes prevent the access of biomolecular devices that have not been appropriately designed. Therefore, the creation of biomolecular devices that are sensitive to the intracellular environment has been suggested as a method to overcome these physiological bottlenecks.

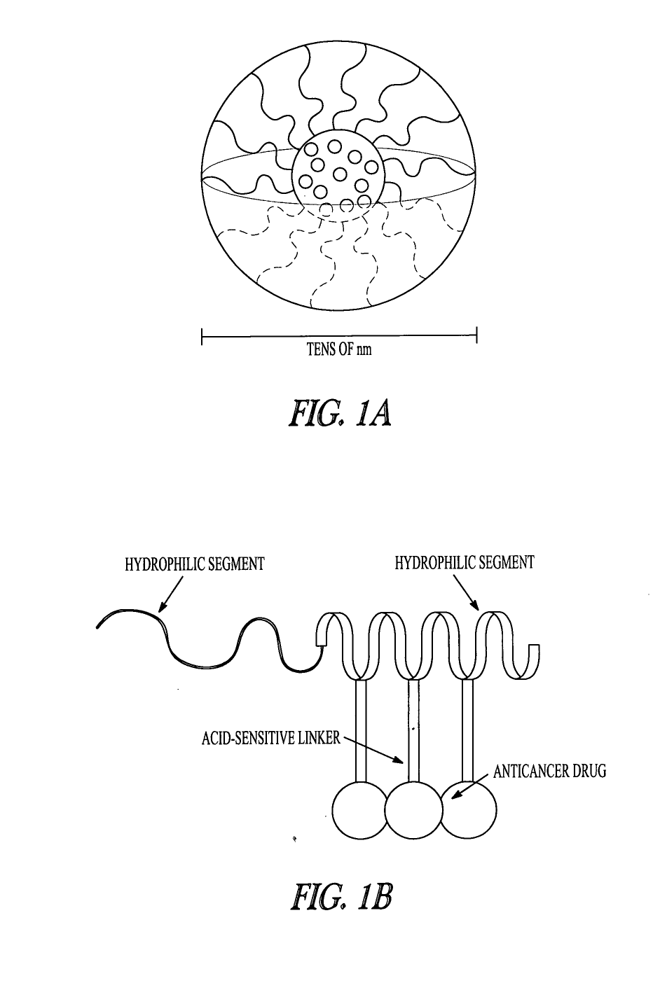

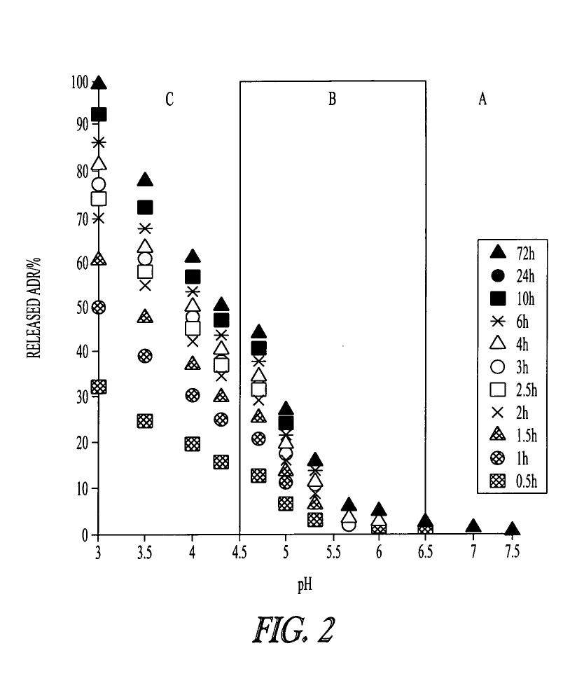

From self-assembling acid-sensitive amphiphilic block copolymers we have prepared a polymeric micelle that is activated by the intracellular pH value (FIG. 1). The polymeric micelle is a supramolecular assembly with characteristic properties, such as a core-protecting double-layer structure that is tens of nanometers in diameter, low toxicity in the human body, and has a prolonged circulation in the blood owing to its high water-solubility, thus avoiding phagocytic and renal clearance. In addition, the functionality of the micelles can be modified simply by changing the chemical structures of the block copolymers, and materials such as drugs, proteins, and DNAs, can be selectively delivered to solid tumors in the body.

Site specific tumor targeting in the body is achieved by the enhanced permeability and retention (EPR) effect, proposed by Maeda and Matsumura. According to their report, solid tumors have abnormal blood vessels with loose junction and insufficient lymphatic drainage, so that the micelles easily escape from the blood vessel and accumulate in tumor tissues but they hardly return to the blood stream again. In general, cells take up large materials, such as the micelles, by folding the cell membrane inwardly, surrounding the materials to be ingested.

The material is then engulfed in small bubble-like endocytic vesicles. This is called the endocytosis process that allows supramolecular assemblies to sneak into intracellular regions avoiding the cell-membrane transporters. After the micelles are taken up to the cell interior through endocytosis, the substance transport occurs. The endocytic vesicles change from early and late endosomes and finally to lysosomes in which the proton concentration is 100-times lower (pH 5.0) than the physiological condition (pH 7.4), which is an important in vivo chemical stimuli that can be used to trigger functional biomolecular devices. Release of the therapeutic agent at the lower pH is illustrated in Scheme 1-1, demonstrating a pH-controlled drug release.

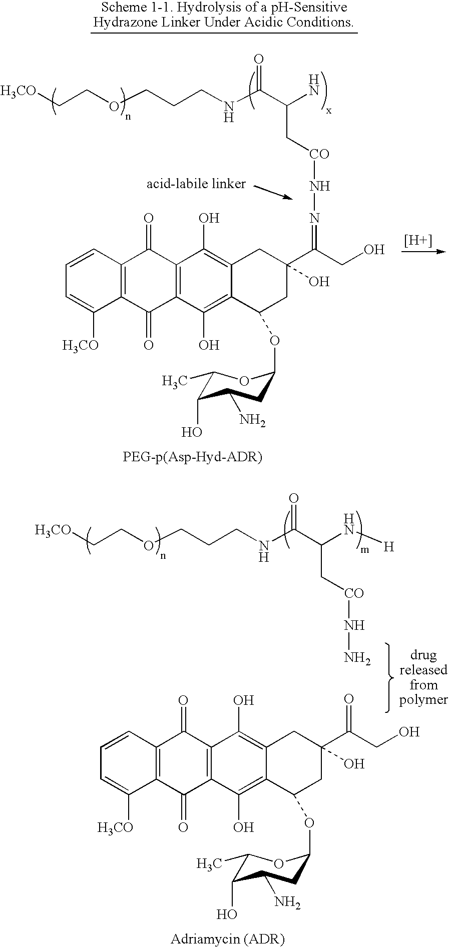

An amphiphilic block copolymer, poly(ethylene glycol)poly(aspartate-hydrazone-adriamycin) (PEG-p(Asp-Hyd-ADR)), was synthesized using the aspartic acid of poly-(ethylene glycol)poly(b-benzyl-1-aspartate) (PEG-PBLA) as a convenient template (See Example 1 above). A Schiff base was formed between the C13 ketone of ADR and the hydrazide groups of the PEG-p(Asp-Hyd) block polymer. This linker is effectively cleavable under acidic conditions at around pH 5.0, which correspond to that of lysosomes in mammalian cells.

The PEG-p(Asp-Hyd-ADR) block copolymer prepared from PEG-PBLA can be a polymer of formula I:

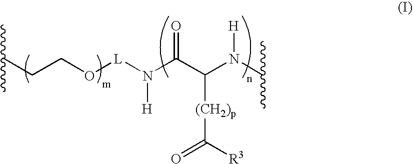

where the applicable values for m, n, p, L, and R³ are as defined in the specification above. For example, polymers of formula I have been prepared wherein about 80% of the R³ groups are —NH—N=[drug], wherein the drug is doxorubicin for some R³ groups and wortmannin for other R³ groups. Substantially all of the remaining R³ groups are hydroxyl groups, however some may be benzyloxy groups. Additional manipulations can be carried out on the carbonyl and R³ moiety to provide an R³ that is a hydroxyl protecting group (“PG”), —O-PG, such as an acetyl group, an alkyl ester group, or other groups such as those described in the section above on protecting groups. The hydrazine groups can be installed by the methods described by Bae et al. (Angew. Chem. Int. Ed., 2003, 42, 4640-4643) or they can be installed by aminolysis of the benzyloxy group using anhydrous hydrazine. The latter technique aids in not only controlling, but also in increasing, the substitution ratio of the hydrazide moiety.

PEG-PBLA was synthesized from the ring-opening polymerization of β-benzyl-1-aspartate N-carboxy-anhydride (BLA-NCA). Polymerization of BLA-NCA was initiated by the terminal primary amino group of α-methoxy-ω-amino poly(ethylene glycol) under argon atmosphere in distilled dimethylformamide to provide the PEG-PBLA. The benzyl groups of PEG-PBLA were substituted with hydrazide groups for drug binding by ester-amide exchange (EAE) aminolysis reaction. PEG-PBLA (500 mg) was dissolved in 10 mL of dry DMF, and anhydrous hydrazine (0.62 mg, MW=32.05) was added to the solution. The reaction was allowed to proceed at 40° C. for 24 hours, followed by the deprotection of remained benzyl groups with 0.1N NaOH in water at 25° C., followed by dialysis against 0.25% ammonia solution.

After freeze-drying, the PEG-p(Asp-Hyd) (50 mg) obtained was dissolved in 10 mL of DMSO, and an excess amount of ADR-HCl, with respect to the drug-binding hydrazide residues of the polymer side chains, was added. The mixture was stirred at about 23° C. for 3 days while being protected from light, followed by gel purification using Sephadex LH-20 to completely remove unbound ADR. Purified PEG-p(Asp-Hyd-ADR) was dissolved in DMSO again to prepare micelles by a dialysis method.

Adriamycin (ADR) was then conjugated to the polymer backbone through an acid-labile hydrazone bond between C13 of ADR and the hydrazide groups of the PEG-p(Asp-Hyd) block copolymer. Subsequently, the polymeric micelles were prepared by a dialysis method which brought the organic components into an aqueous environment. The micelles were about 65 nm in diameter and of uniform size, as confirmed by dynamic light-scattering measurements (DLS). ADR is an anticancer agent and suppresses cell growth by binding with DNA strands in the cell nucleus. Despite its efficacy, ADR use is frequently accompanied by toxic side effects. However, its activity is suspended by binding to materials such as polymers, antibodies, and molecular complexes. In addition, the detectable fluorescence of ADR allows it to be used as a fluorescence probe in this Example.

The acid-sensitivity of the micelles was evaluated by reversed-phase liquid chromatography (RPLC). As shown in FIG. 1-2, the micelles release ADR both time- and pH-dependently as the pH value decreases from pH 7.4 to 3.0. The micelles were stable over 72 hours in region A (FIG. 2), which corresponds to physiological and early endosomal conditions. On the other hand, the release of ADR gradually increases and reaches equilibrium as the pH decreases in regions B and C. The ADR release profile in region B is notable considering that the pH values in late endosomes and/or lysosomes in the cells are around 5.0 where the acid-sensitive hydrazone bonds can be cleaved most effectively. Because the formation of reversible hydrazone bonds is hindered by strong acidity, the loading content of ADR on the micelles was calculated from the maximum ADR release at pH 3.0 in region C The calculation revealed that the micelles consisted of the block copolymers containing ADR with 67.6% mol substitution with respect to aspartate units of PEG-p(Asp-Hyd-ADR).

Measurement of fluorescence intensity reveals that the micelles are stable under physiological conditions and fluorescence only occurs when the ADR is released under acidic conditions. The micelles and free ADR were incubated in cell culture medium, Dulbecco's Modified Eagle's Medium (DMEM) supplemented with 10% fetal bovine serum for 24 hours. Ion and pH levels are controlled in DMEM, which is very similar to physiological condition in the body. Concentrations of ADR and the ADR bound in the micelles were adjusted to be equivalent (100 μg mL⁻¹).

Samples were excited with the wavelength of 485 nm, and the fluorescence at 590 nm was monitored by a spectrofluorometer. Compared with the intense fluorescence of free ADR, the fluorescence intensity of the ADR-bound in the micelles remained low and no significant change in intensity was observed after 24 hours of monitoring. Like most fluorescence materials, the fluorescence of ADR is quenched in a high concentration in solution. This phenomenon also occurs in the micelle core where ADR molecules are confined at high local concentrations. The fluorescence remains quenched as long as the ADR is incorporated in the micelle core and a change in fluorescence reflects the, release of the ADR from the micelles. Thus, the pH sensitive structural change of the micelles can be detected through the change in fluorescence.

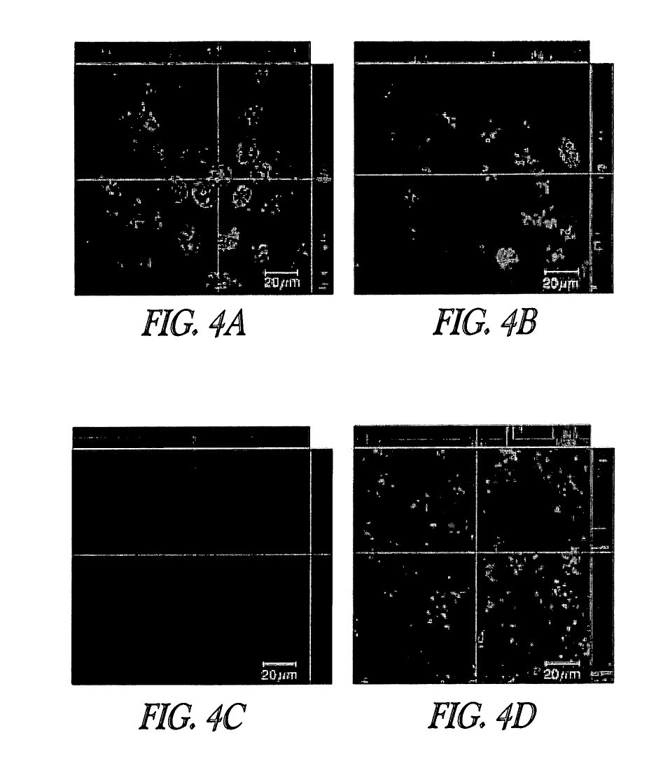

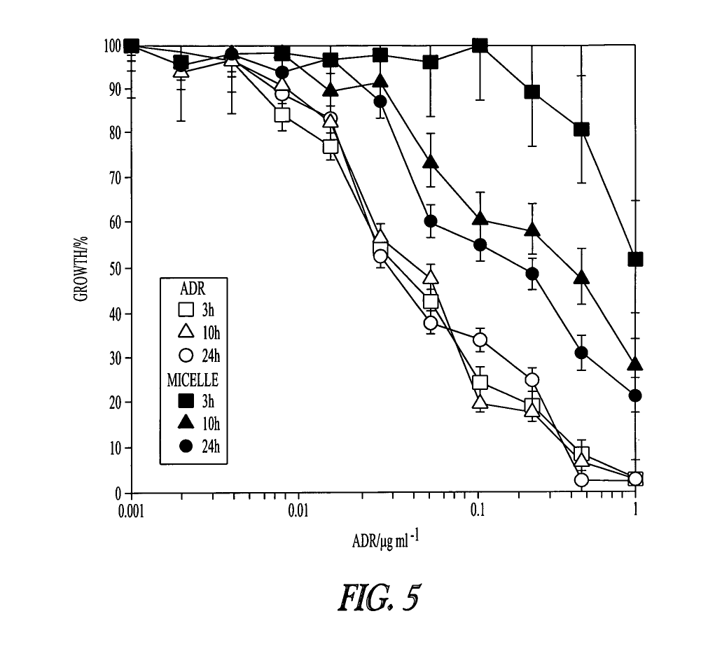

Observations using confocal laser scanning microscopy (CLSM) reveal the intracellular localization of micelles that were incubated with human small cell lung cancer SBC-3 cells. As shown in FIG. 4, a time-dependent fluorescence change in intensity was observed over 24 hours. After 1 hour exposure, an increase in fluorescence intensity was observed for SBC-3 incubated with ADR (FIG. 4a), but no such increase was detected with the micelles (FIG. 4c). On the other hand, a considerable fluorescence change was observed in the cells exposed to the micelles after 24 hours incubation (FIG. 4d), which clearly demonstrates intracellular distribution of the micelles and the released ADR.

Compared with FIG. 4b, which shows that ADR is only accumulated in cell nuclei, FIG. 4d indicates that the localized fluorescence is dot-shaped within the cytoplasm suggesting the presence of the micelles trapped in the endocytic vesicles. In general, it is very difficult to distinguish between the fluorescence material ADR and its polymer conjugates in solution because both exhibit intense fluorescence. However, the micelles solve this problem because of their characteristic fluorescence quenching effects.

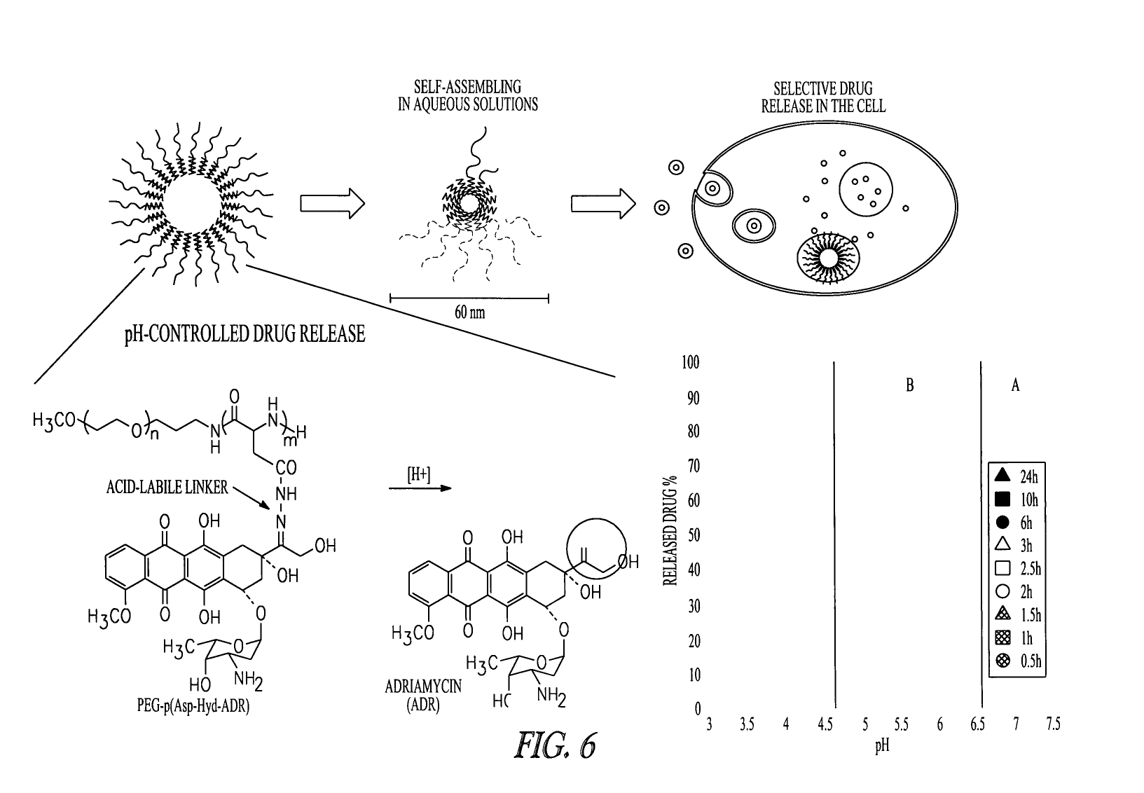

As a system releasing bioactive molecules, the micelles are required to maintain the ability of the loaded ADR to suppresses cell growth by binding with DNA strands in the cell nucleus. FIG. 5 shows the growth-inhibition effects of the micelles on SBC-3 cells. The results obtained with the micelles gradually approach those of free ADR, which demonstrates that the ADR released from the micelles is pharmaceutically active. Therefore, one can conclude that ADR accumulates in the cell nuclei after release from the micelles localized within the cytoplasm.

The pH-sensitive drug release from polymeric micelles in intracellular acidic regions of a cell, according to an embodiment of the invention, is illustrated in FIG. 6. When a single type of drug is linked to a polymer chain, as in FIG. 6, it is understood that while only one type of drug is shown, the micelle will be either a physically mixed micelle or a simply mixed micelle, so that combination drug therapy can be carried out. Additionally, various chemically mixed micelles can be prepared by replacing one or more of the doxorubicin moieties with other drug conjugate moieties, as described herein.

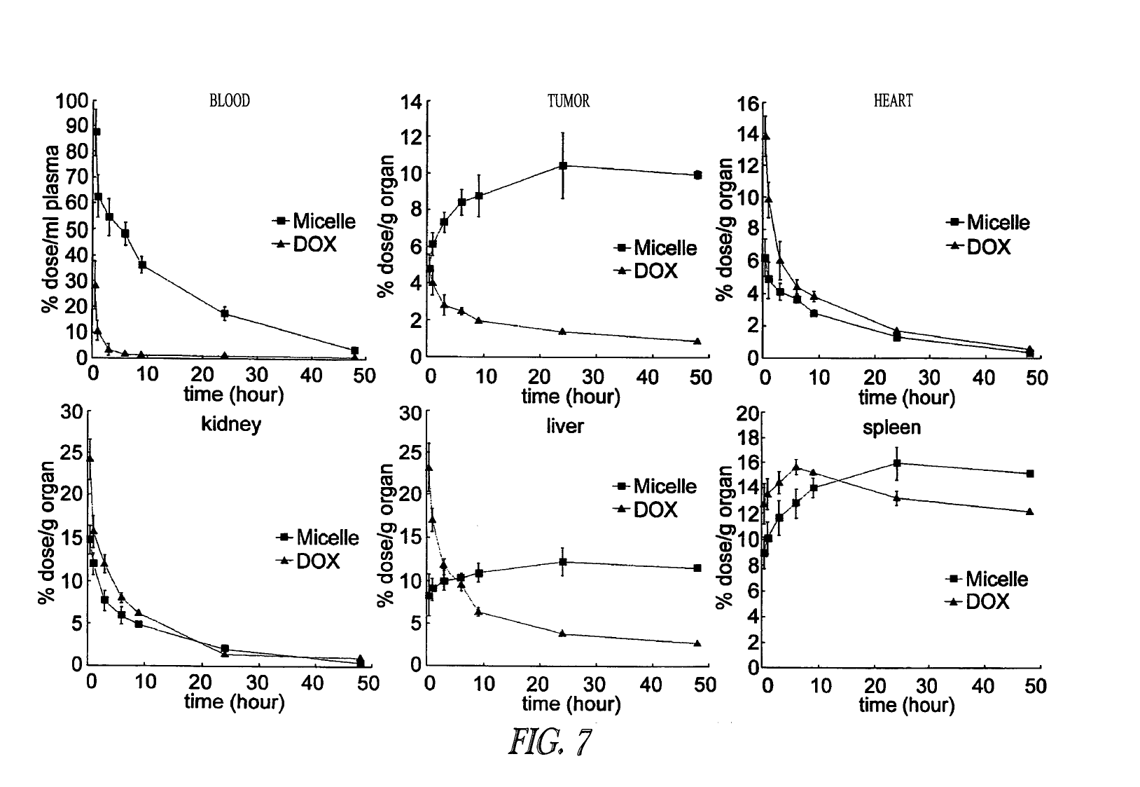

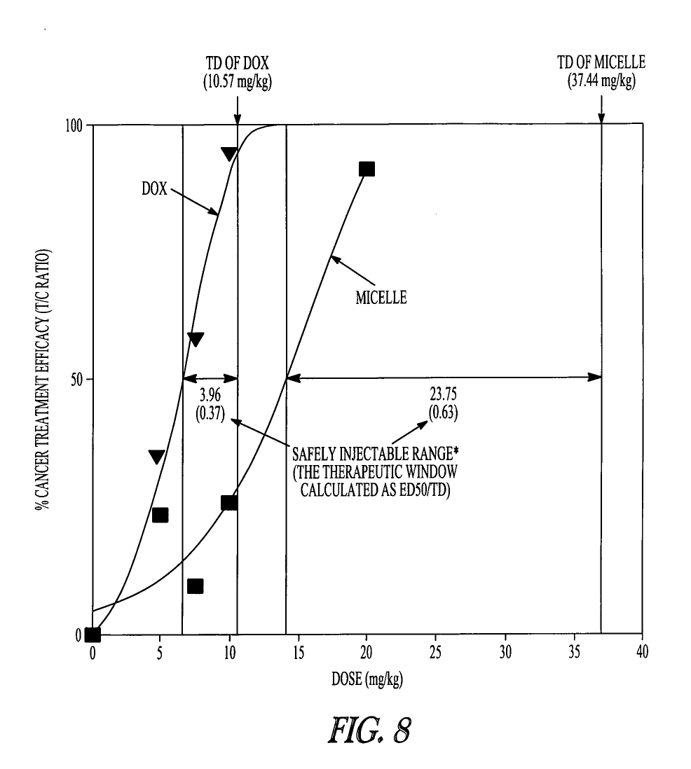

FIG. 7 illustrates biodistribution and tumor specific accumulation of micelles of the invention, and a comparison of plasma levels of doxorubicin and polymer-linked doxorubicin delivered in the micelles described herein, according to an embodiment of the invention. Animal studies confirmed the prolonged circulation in the blood and tumor-specific accumulation of the pH-sensitive micelles. The CDF1 mice (female, n=6), when the tumor volume reached about 100 mm³, were injected with DOX or the micelles in a volume of 0.1 mL/10 g body weight for the experiments. The dose was either 10 mg/kg for DOX or the micelles (DOX equivalent).

After the injection, blood, tumor and major organs (heart, kidney, liver and spleen) were collected at 0.5, 1, 3, 6, 9, 24 and 48 hours, followed by HPLC analysis (see Supporting Information of Bae et al., Journal of Controlled Release 122 (2007) 324-330 for related protocol details, incorporated herein by reference). As can be seen in the figure, 17% of the dose of micelles remained in the blood after 24 hours, while almost none of the dose of doxorubicin remained after only 10 hours. Similarly, an 11-fold higher accumulation of micelles were found in tumors after 48 hours.

Table 2 illustrates a data comparison of cure rates between mice treated with conventional doxorubicin treatment techniques and those treated with polymer-doxorubicin micelles.

TABLE 2

| | Dose | Weight change | Toxic | Complete |
|---|---|---|---|---|
| Sample | (mg/kg) | on day 30 (%) | Death | Cure |
| Control | 0 | −2.18 ± 1.74 | 0/6 | 0/6 |
| DOX | 5 | −13.35 ± 0.59 | 0/6 | 0/6 |
| | 10 | −16.84 ± 1.26 | 0/6 | 1/6 |
| DOX-Micelle | 5 | −0.89 ± 1.68 | 0/6 | 0/6 |
| | 10 | −4.51 ± 1.44 | 0/6 | 0/6 |
| | 20 | 3.13 ± 1.60 | 0/6 | 2/6 |
| | 40 | −4.07 ± 0.92 | 0/6 | 3/6 |

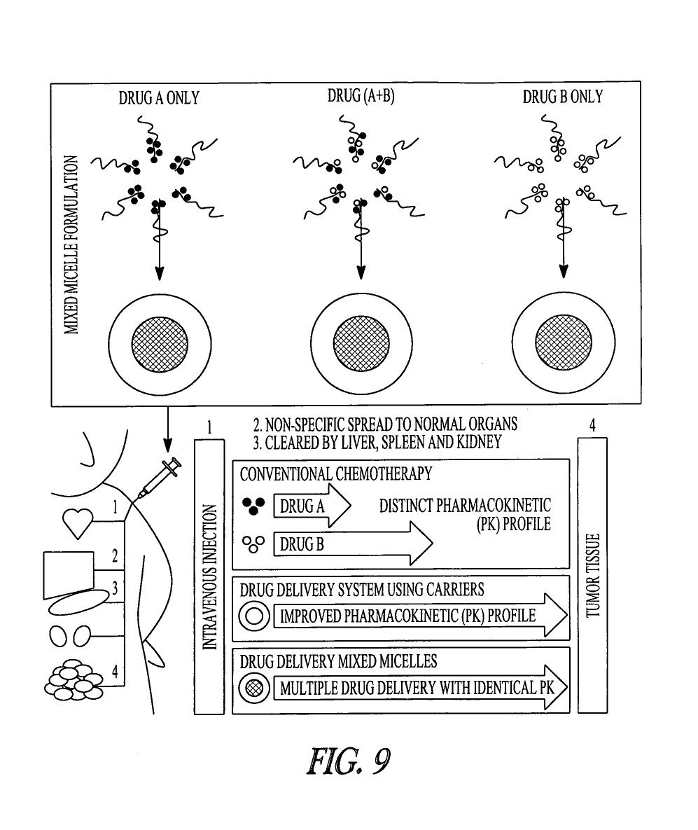

As can be seen from Table 2 and FIG. 8, the micelles of the invention provide a broader therapeutic window than standard administration, based on treatment-to-control (T/C) ratio.

Cancer treatment efficacy of the pH-sensitive micelles was evaluated by comparing the therapeutic windows of small molecule drugs (doxorubicin) and the doxorubicin-conjugated micelles. Therapeutic windows were determined based on the ratio of ED₅₀ to TD. ED₅₀ and TD are defined as the effective dose that induces 50% decrease in tumor volume and the toxic dose that reduces 20% of body weight of mice, respectively. The data show the dose range in which each sample can be safely injected while achieving effective cancer treatments.

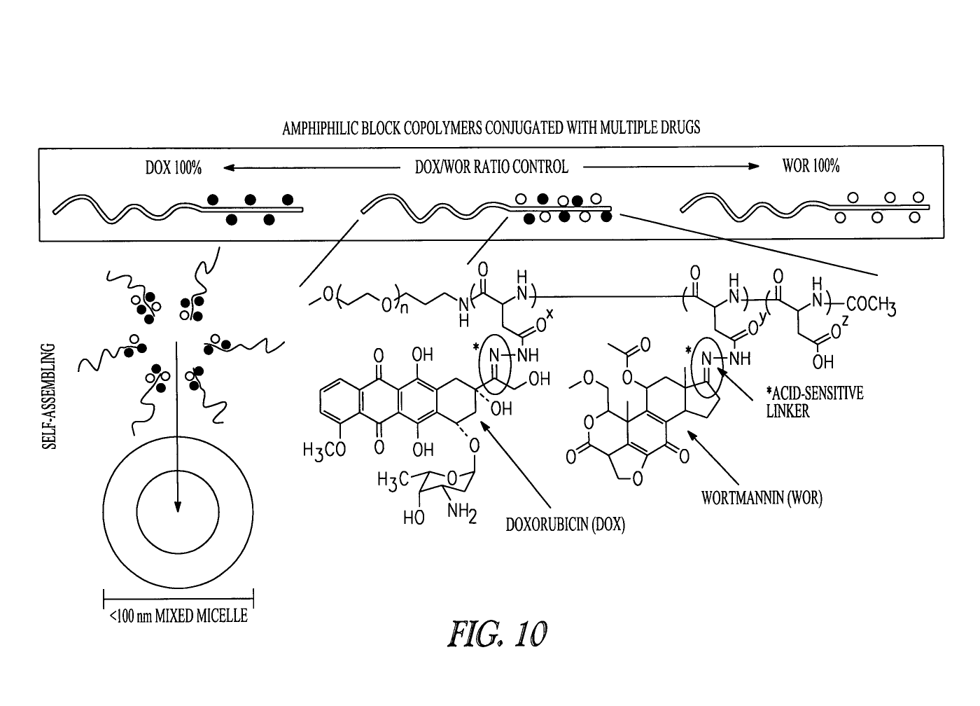

FIG. 9 illustrates the improved effectiveness of combination chemotherapy using mixed micelles as a result of drug accumulation in a cancerous tumor. Initial drug mixing ratio at injection can be preserved within the tumor tissue because the mixed micelles can deliver multiple drugs at the same pharmacokinetic profiles. The systemic drug concentration of drugs injected according to conventional chemotherapy is often significantly reduced by the liver, spleen, and kidneys before reaching the patient's tumor. Known micelle carriers are designed to deliver only one type of drug and may not sufficiently accumulate in tumors. Using the mixed drug micelles of the invention, significant synergistic and combination effects of chemotherapy on cancer treatment are expected.

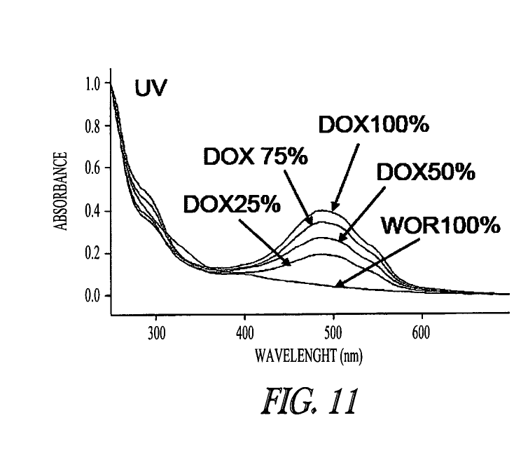

FIG. 10 illustrates mixed micelles for multiple drug delivery, according to various embodiments of the invention. The schematic illustrates the ‘tunability’ of the polymers of various embodiments, wherein any percentage from about 0.1% to about 99.9% of one drug can be prepared, while the balance of drugs linked to the polymer chain are a different drug conjugate. In this figure, a polymer with varying ratios of doxorubicin and wortmannin from 100:0 to 0:100 are schematically illustrated. Other carbonyl containing drugs, or drugs with appropriate linkers, can be exchanged for either of, or both, doxorubicin and wortmannin, in various embodiments of the invention.

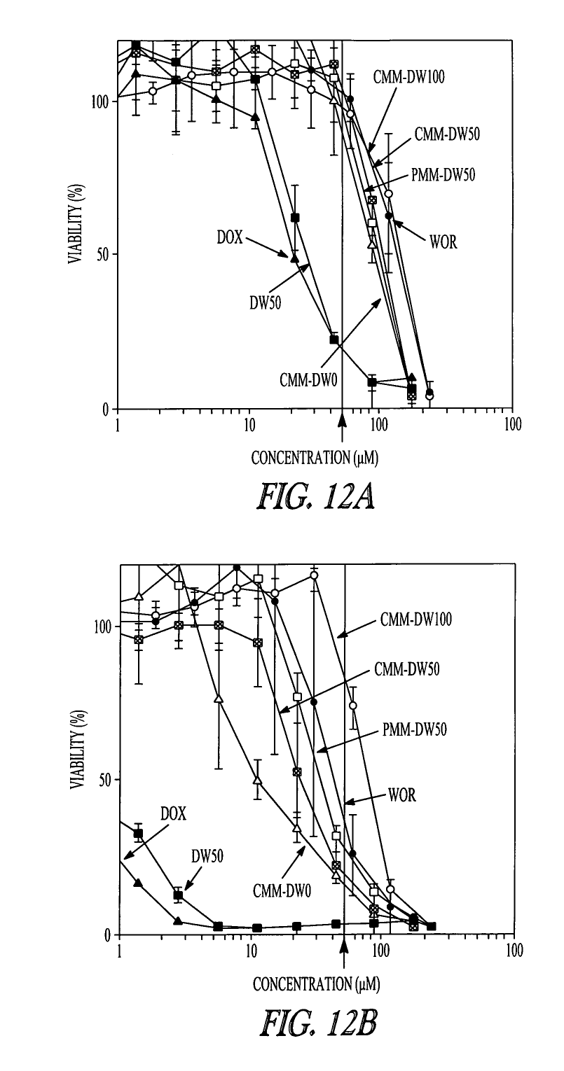

In one specific embodiment, a dual drug delivery polymer including a doxorubicin conjugate (“DOX”) and a wortmannin conjugate (“WOR”) on the same polymeric chain can be used to prepare chemically mixed micelles for combination therapy. FIG. 11 illustrates UV absorbances of five polymer-drug bioconjugates that have been prepared, namely 100% DOX, 75% DOX/25% WOR, 50% DOX/50% WOR, 25% DOX/75% WOR, and 100% WOR.

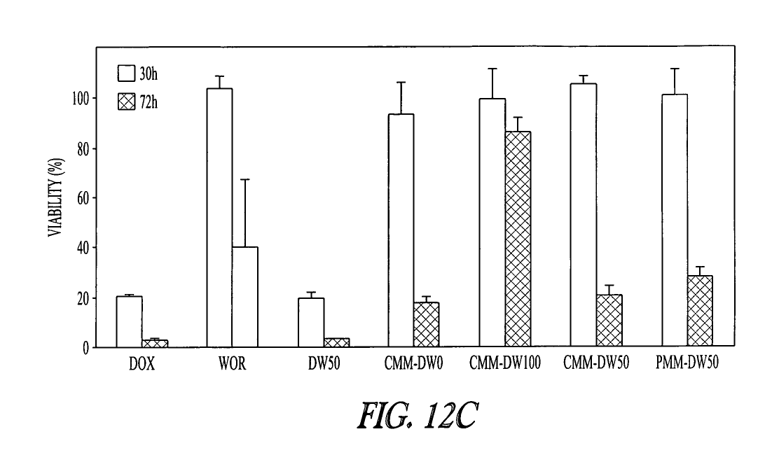

FIG. 12 illustrates in vitro data for DOX/WOR micelle formulations. The compositions for the mixed polymeric micelles are distinguished with the names ‘chemically mixed micelle (CMM)’ and ‘physically mixed micelle (PMM)’ depending on how mixed micelles were prepared. For example, when a mixed polymeric micelle was formed from the block copolymers that contain both DOX and WOR on a single polymer chain simultaneously, it is a CMM. In contrast, PMM indicates a polymeric micelle that was prepared from two different block copolymers, containing only DOX or WOR respectively. Cytotoxic activity of combination use of free drugs and mixed polymeric micelles against a human breast cancer MCF-7 cell line at 30 hours (A) and 72 hours (B) after drug exposure. The difference in cellular viability was compared with 50 μM drug concentration (C). See Bae et al., Journal of Controlled Release 122 (2007) 324-330, which is incorporated herein by reference.

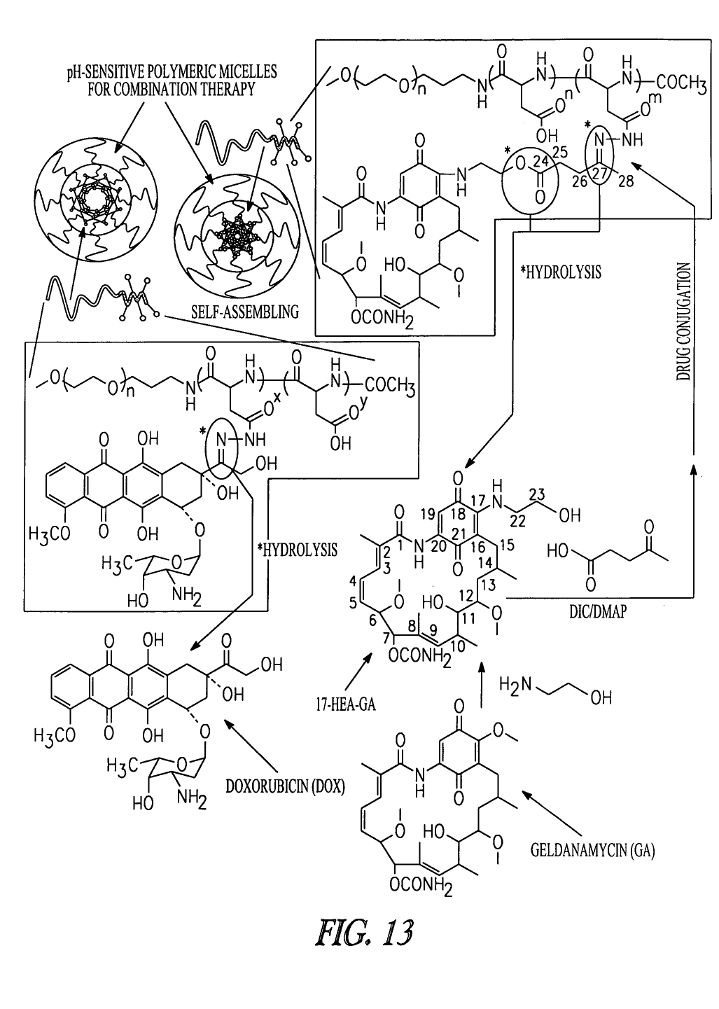

FIG. 13 illustrates examples of DOX/GA mixed micelle formulations. Chemical design and preparation of pH-sensitive polymeric micelles. HSP90 and TOPOII inhibitors have been conjugated to a poly(ethylene glycol)-poly(aspartate-hydrazide) block copolymer through degradable hydrazone linker for pH-responsive drug release control.

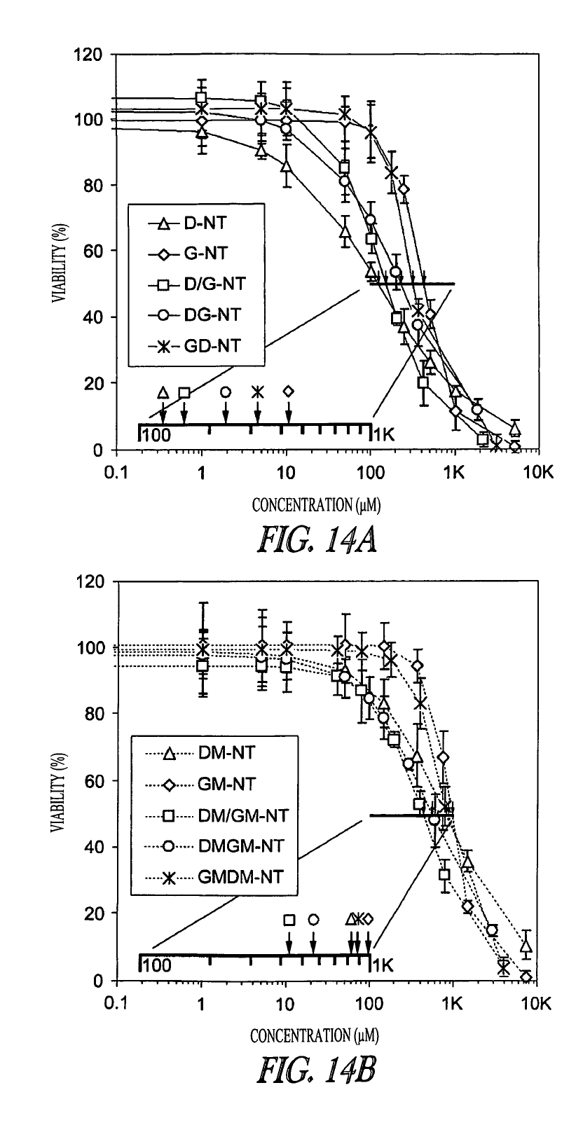

FIG. 14 illustrates the viability of MCF-7 treated with small molecule drugs (A) and micelles (B) through different regimen schedules and combination formulation at normothermia (37° C.). D, G, DM, GM and NT stand for DOX, 17-HEA-GA, DOX-loaded micelle, 17-HEA-GA-loaded micelle, and normothermia, respectively. Regimen schedules for small molecule drugs (or polymeric micelles) are described as follows: D(DM)-NT: add D(DM) alone; G(GM)-NT: add G(GM) alone; D/G(DM/GM)-NT: add D(DM) and G(GM) simultaneously; DG(DMGM)-NT: add D(DM) first and G(GM) after 24 hours; GD(GMDM)-NT: add G(GM) first and D(DM) after 24 hours. (mean ±SD, n=4)

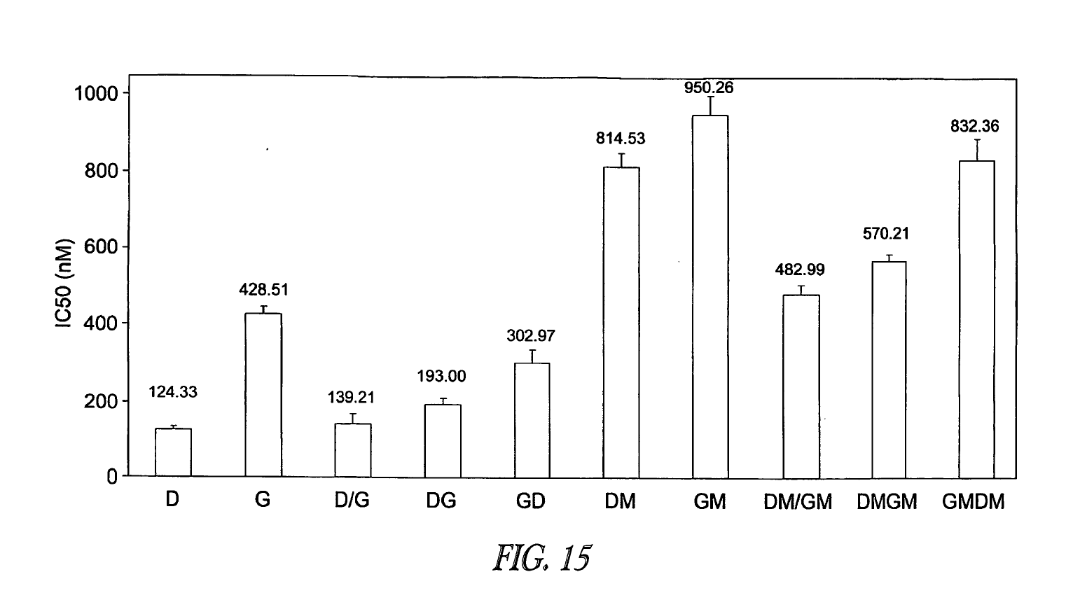

FIG. 15 illustrates a comparison of inhibitory concentrations for suppressing 50% cell viability (IC₅₀) for small molecule drugs and polymeric micelles at normothermia (37° C.). D, G, DM and GM stand for DOX, 17-HEA-GA, DOX-loaded micelle and 17-HEA-GA-loaded micelle, respectively. Regimen schedules for small molecule drugs (or polymeric micelles) are described as follows: D(DM): add D(DM) alone; G(GM): add G(GM) alone; D/G(DM/GM): add D(DM) and G(GM) simultaneously; DG(DMGM): add D(DM) first and G(GM) after 24 hours; GD(GMDM): add G(GM) first and D(DM) after 24 hours. (mean ±SD, n=4).

The in vitro data of synergistic drug ratios obtained from analysis of the mixed micelles of the invention can then be translated into improved anticancer combination therapies in which the desired drug ratio can be controlled and maintained following administration in vivo, so that the synergistic effects can be exploited. Suitable techniques for the translation of the in vitro data to in vivo therapies have been described by Mayer and Janoff (Molecular Interventions (2007), 7(4), 216-223).

In summary, the intracellular localization of pH-sensitive polymeric micelles whose functions are controlled by live cells has been successfully carried out. As a multifunctional biomolecular device, the micelles undergo dynamic changes in structure and/or function in response to environmental stimuli (pH value). Furthermore, the ADR released from the micelles fluoresces, which allows its localization within the living cells to be detected. CLSM reveals that the micelles are trapped in lysosomes where they are programmed to function by responding to low pH, and the released ADR accumulates in the cell nuclei and effectively suppresses the synchronizing cell viability of cancer cells. Thus highly controlled functional biomolecular devices are now available.

Cytotoxicity results have been obtained for the combination of doxorubicin and a geldanamycin analogue, provided as a simply different micelle formulation. The cytotoxicity results on the combination indicate additive or synergistic effects at a one to one drug ratio. Other drug ratios are believed to be able to provide even greater synergistic effects. It is believed that this drug combination can achieve an additive or synergistic anti-tumor efficacy in a murine tumor model.

### Example 3

### Therapeutic Agent Linkages

Reference is made to FIGS. 5-16, where certain aspects and embodiments of the invention are illustrated. It should be noted that in the figures, doxorubicin and doxorubicin conjugates may be illustrated, but the doxorubicin may be exchanged with many other carbonyl-containing anticancer agents, for example, apicidin, cucurbitacin, radicicol, and wortmannin, to name a few, which are also illustrated in Scheme 3.1 below.

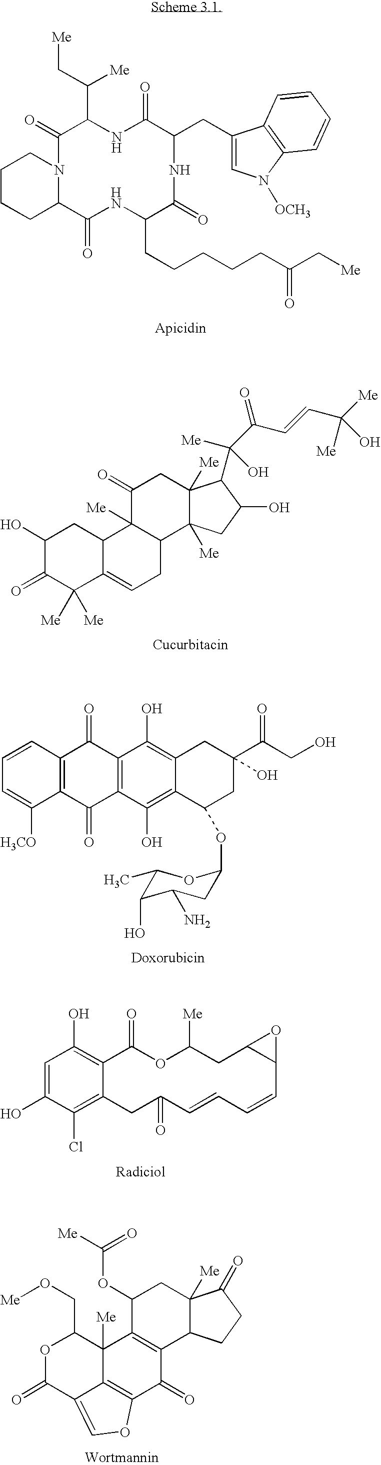

Each of the therapeutic agents illustrated in Scheme 3.1 has accessible and reactive ketones and can be directly condensed with a hydrazide terminated side chain of a polyamide polymer as described herein. Certain therapeutic agents, such as 17-allylamino-17-demethoxygeldanamycin (17-AAG) and paclitaxel, require minor chemical modifications to provide a linker that can link the drugs to hydrazones of polyamide side chains. For example, the macrolide 17-AAG, illustrated below, bears a urethane group at C7. The amine of the group may be sufficiently reactive to form a mixed amide with an activated electrophile, such as an acid chloride, triflate, or the like.

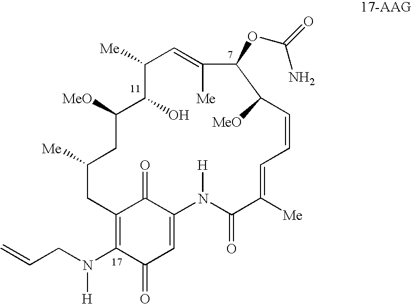

Alternatively, the hydroxyl at C11 may be sterically accessible and could be used to prepare an ester linkage with any suitable acid or acid chloride. Several other suitable transformations are discussed above in the Detailed Description for geldanamycin. For example, Table 1 illustrated that geldanamycin can be substituted with a hydroxyethyl amine linker. The terminal hydroxyl group is not, however, a suitable group for condensing with hydrazone moieties. Scheme 3.2 below illustrates the facile steps that can be taken to convert the HEA-GA derivative to an analog with a suitably reactive carbonyl, the carbonyl of a levulinic acid group.

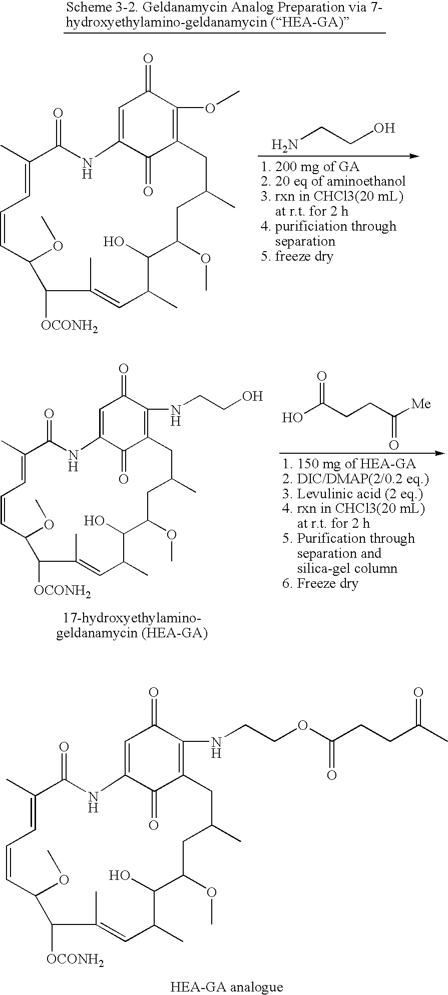

In Scheme 3.2, the free hydroxyl group of HEA-GA was esterified with levulinic acid (4-oxopentanoic acid). Likewise, for drugs such as paclitaxel and triptolide, linkers can be installed by simple esterification of a free hydroxyl with a suitable keto acid, such as levulinic acid. Levulinic acid has been used to prepare analogs for linking both paclitaxel and triptolide to polyamides through hydrazides.

The synthesis of paclitaxel (“PAX”)-linker derivatives, using a levulinic acid linker and a 4-acetyl benzoic acid linker, is illustrated below in Schemes 3.3 and 3.4, respectively.

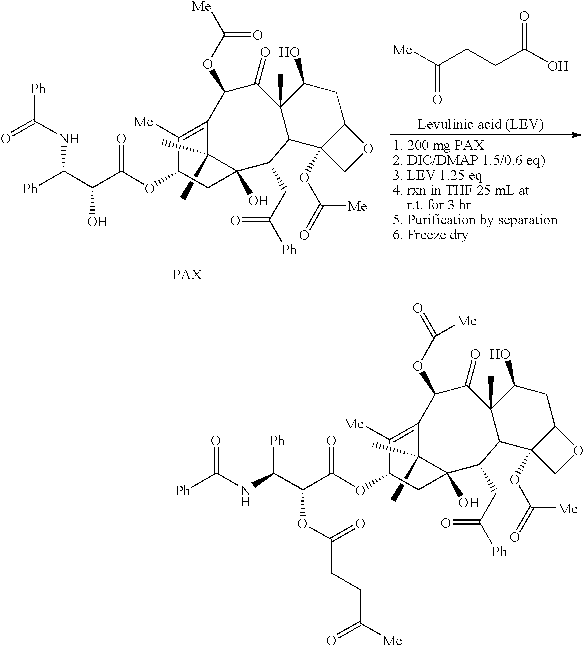

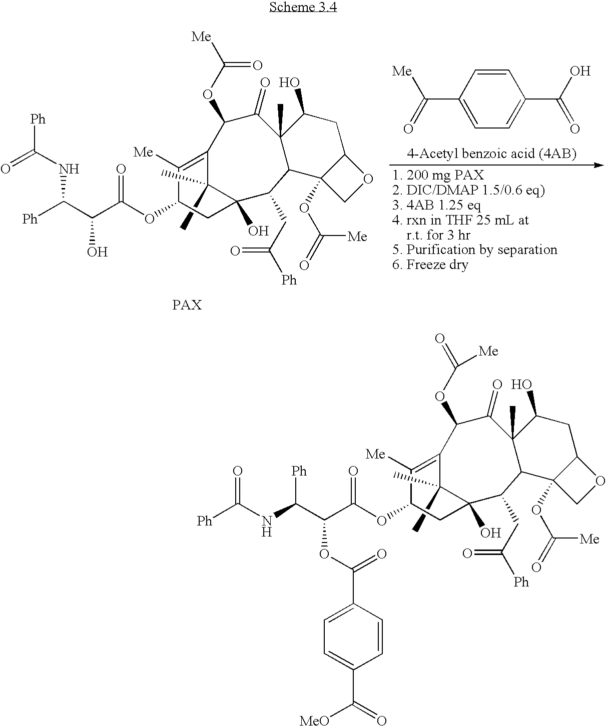

Similar synthetic steps can be used to prepare the levulinic acid ester of triptolide, illustrated in Scheme 3.4 below.

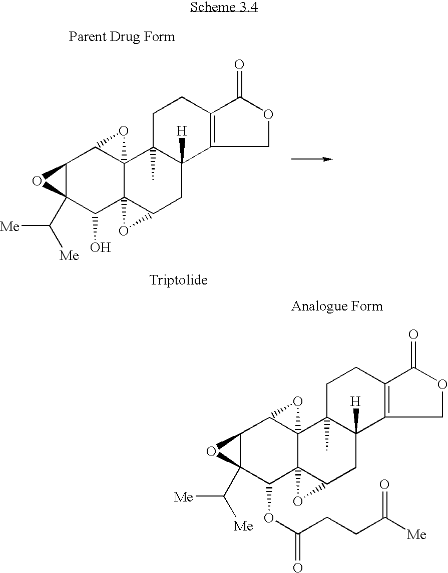

Examples for doxorubicin, geldanamycin, paclitaxel, radicicol, triptolide, and wortmannin drug conjugates have been synthesized and characterized by ¹H NMR and dynamic light scattering measurements (to determine size). These drug conjugate polymers can be used to prepare simply different micelles or physically mixed micelles, such as the combination of doxorubicin and a geldanamycin analogue. All of these cases afford novel solubilized drug combination formulations for combination therapy, especially suitable for intravenous administration.

### Example 4

### Drug Linked Polymers

Many chemotherapeutic agents with low water solubility can be advantageously delivered to tumor cells using the polymer micelles of the invention. Many therapeutic agents have suitable carbonyl functionalities to link them to hydrazone side chains of the polyamide block copolymers. Other therapeutic agents can be linked to the hydrazone moieties by using a linking group that has a ketone or aldehyde group in the linker, and an appropriate functionality that can be used to link one end of the linker with a hydroxyl, carboxyl, or other functional group of the agent.

For example, Scheme 4.1 below illustrates the preparation of a polymer linked to geldanamycin through an ester linker. Similar techniques can be used to link other therapeutic agents to the hydrazide side chains of the polyamides for preparing the micelles of the invention.

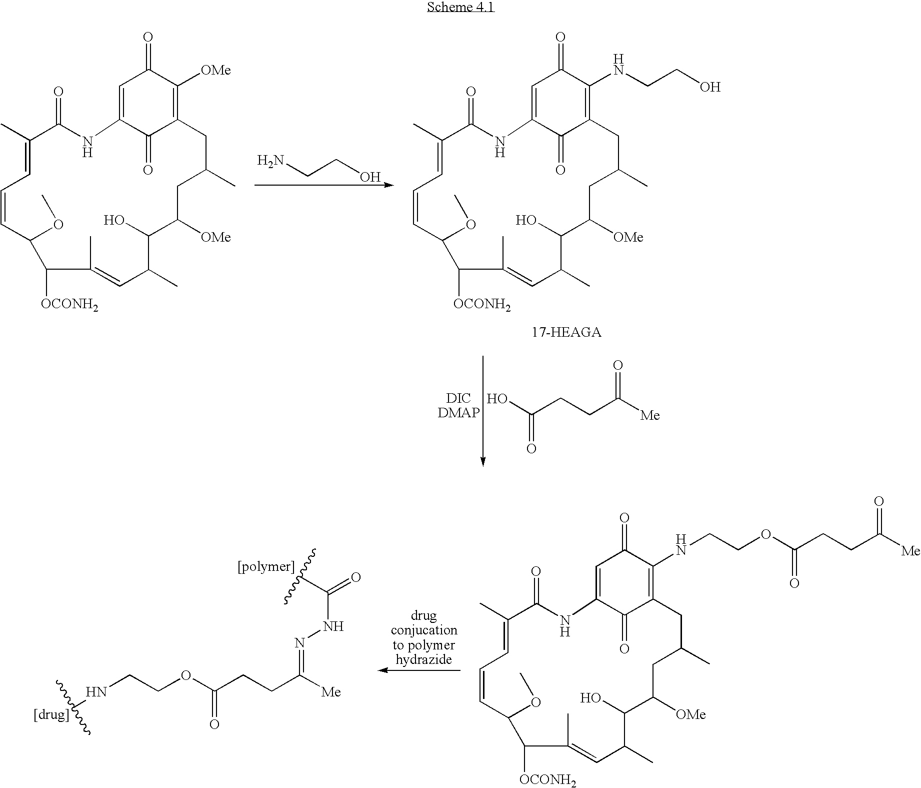

The ester linkage technology can be used to provide a carbonyl ‘handle’ for many therapeutic agents, such as those with a hydroxyl or carboxy functionality.

### Example 5

### Polymers for Preparing Mixed Micelles

A specific advantage of the combination drug delivery micelle formulations described herein is that they do not aggregate in water, which is a problem encountered when attempting to combine poorly water-soluble drugs together for simultaneous intravenous drug administration. Lipophilic drugs are solubilized by various ways, such as pH adjustment, cosolvents, surfactants, and complexes. However, adding a lipophilic drug to other drugs, and thus other excipients, can result in precipitation. Accordingly, current combination therapy approaches would require multiple IV catheter lines for the administration of multiple anti-cancer drugs. By using the micelles described herein, precipitation of the drug combinations is not an issue and the need for multiple IV catheter lines is eliminated.

A variety of therapeutic agents can be linked to the polymer chains described herein to prepare simply mixed micelles, physically mixed micelles, and chemically mixed micelles (see FIG. 3). Table 3 shows five specific examples of drugs that can be used in various embodiments of the invention, in any combination.

TABLE 3
Six drug examples used as mixed micelle formulation.

| Drug Name | Abbreviation | Therapeutic Target and Action |
|---|---|---|
| Doxorubicin | D | Topoisomerase II inhibition |
| Wortmannin | W | Phosphoinositide 3-kinase |
| | | inhibition |
| 17-Hydroxy- | G or | Heat Shock Protein 90 inhibition |
| ethylamino-17- | 17-HEA-GA | |
| dimethoxy- | | |
| geldanamycin | | |
| Triptolide | T | Heat Shock Protein 70 inhibition |
| 2-Methoxy-Estradiol | M | Caspase-3 activation and apoptosis |
| | | as a result of oxidative stress or by |
| | | action on microtubules |
| Paclitaxel | P | Microtubule growth interference |

Table 4 further illustrates the variety of combination drug therapy strategies that can be used with the drugs listed in Table 3. This approach can be extended to all other therapeutic agents, as described herein.

TABLE 4
Drug Combinations.

| | Examples of Chemotherapy Drug |
|---|---|
| Type | Combinations |
| single drug | D, W, G, T, M, and P alone |
| 2 drug | DW, DG, DT, DM, DP, WG, WT, WM, |
| combination | WP, GT, GM, GP, TM, TP, MP |
| 3 drug | DWG, DWT, DWP, DWM, DGT, DGM, |
| combination | DGP, DTM, DTP, DMP, WGT, WGM, |
| | WGP, WTM, WTP, WMP, GTM, GMP, |
| | GTP, TMP |
| 4 drug | DWGT, DWGM, DWGP, DWTM, DWTP, |
| combination | DWMP, DGTM, DGTP, DGMP, DTMP, |
| | WGTM, WGTP, WGMP, WTMP, GTMP |
| 5 drug | DWGTM, DWGTP, DWGMP, DWTMP, |
| combination | DGTMP, WGTMP |
| 6 drug | DWGTMP |
| combination | |

A Chemically Mixed Micelle that includes a geldanamycin derivative and doxorubicin conjugate, illustrated below in Scheme 5.1, can be prepared by forming hydrazide bonds to the polyamide polymers.

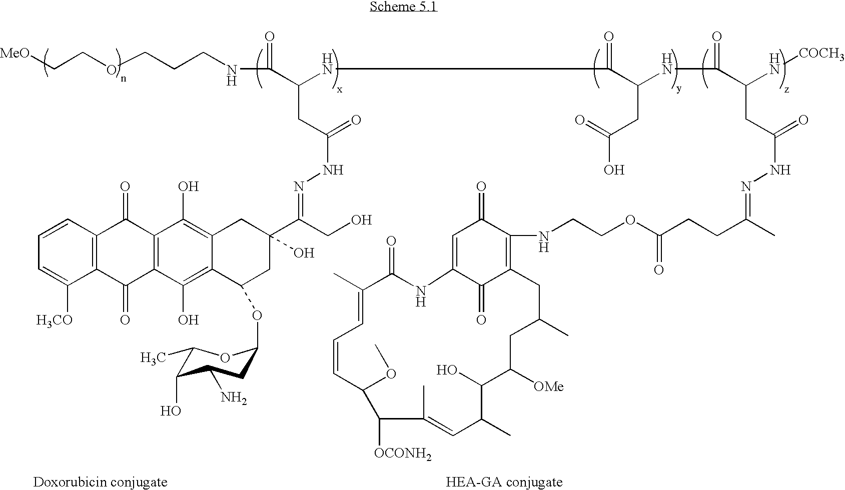

Scheme 5.2 below illustrated an example of polymers that can be used to prepare a Physically Mixed Micelle that includes a doxorubicin conjugate and a geldanamycin derivative conjugate.

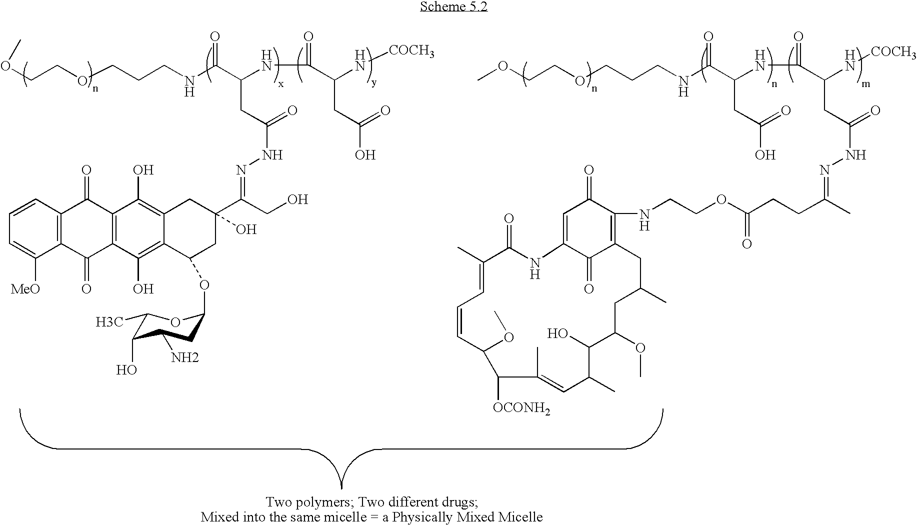

A significant advantage of the Physically Mixed Micelle is that it is a simple matter to vary the ratio of the drugs in the micelle formulation by simply varying the ratio of the doxorubicin conjugate polymer to the geldanamycin derivative conjugate polymer that are added into the micelle preparation mixture. For example, micelles with drug ratios from 1:100 to 100:1 can easily be prepared by adding the appropriate amount of each type of polymer to the preparation.

Mixed micelles that have been prepared include chemically mixed micelles prepared from a polyamide that is linked to both doxorubicin and geldanamycin (see Scheme 5.1 above), and a polyamide that is linked to doxorubicin and wortmannin.

Physically mixed micelles include the combinations of paclitaxel and geldanamycin, paclitaxel and doxorubicin, and paclitaxel and triptolide. Studies to hone and revised the combinations therapy techniques and dosages are currently under way.

All publications, patents, and patent documents cited herein are incorporated by reference, as though individually incorporated by reference. The invention has been described with reference to various specific and preferred embodiments and techniques. However, it should be understood that many variations and modifications may be made while remaining within the spirit and scope of the invention.

What is claimed is:

1. A block polymer comprising a first block and a second block;
wherein
the first block comprises two or more ethylene glycol segments;
the second block comprises two or more amino acid units derived from aspartic acid, glutamic acid, or a combination of aspartic acid and glutamic acid;
two or more amino acid side chains of the second block are individually covalently linked to therapeutic agents through hydrazide moieties; and
the therapeutic agents comprise at least two different therapeutic agents.

2. The polymer of claim 1 wherein the hydrazide moieties are formed from the condensation of side chain carboxylate moieties of the second block, hydrazine or hydrazine derivatives, and carbonyl moieties of the therapeutic agents or carbonyl moieties of a linking group on the therapeutic agent.

3. The polymer of claim 2 wherein the linking group on the therapeutic agent comprises a C₁-C₂₀ carbon chain, ring, or combination thereof, optionally interrupted by one to eight oxygen atoms, nitrogen atoms, or amide groups and optionally substituted with one to eight oxo groups.

4. The polymer of claim 1 wherein the therapeutic agents comprise drugs that are effective for the treatment of cancer and the therapeutic agents have low water solubility.

5. The polymer of claim 1 wherein the eight or more ethylene glycol segments form a poly(ethylene glycol) chain that has a molecular weight of about 400 to about 30,000 g/mol, the poly(ethylene) glycol chain is straight or branched, and the poly(ethylene glycol) chain terminates with a hydroxyl group, an alkoxy group, a hydroxyl protecting group, or an optionally substituted or protected amino group.

6. The polymer of claim 1 wherein one or more amino acid side chains of the second block are individually covalently linked to therapeutic agents through ester linkages.

7. The polymer of claim 1 wherein the first block and the second block are linked to each other through an amide bond or a linking group.

8. The polymer of claim 1 wherein the molecular weight of the second block is about 500 to about 20,000 g/mol, and the amino acid units are optionally derived from L-amino acids.

9. The polymer of claim 1 wherein greater than about 50% of the amino acid side chains are individually linked to therapeutic agents, and the polymer comprises two, three, or four different types of therapeutic agents.

10. The polymer of claim 9 wherein the different therapeutic agents provide a synergistic therapeutic effect when administered to a cancer patient.

11. The polymer of claim 4 wherein the therapeutic agents comprise aclarubicin, apicidin, bortezomib, benzyloxycarbonyl-L-leucyl-L-leucyl-L-leucinal, cyclopamine-KAAD, cucurbitacin, dolastatin, doxorubicin, fenritinide, geldanamycin, herbimycin A, 2-methoxyestradiol, paclitaxel, radicicol, rapamycin, triptolide, wortmannin, or a combination thereof.

12. The polymer of claim 11 comprising a first block and a second block;
wherein
the first block comprises about 10 to about 600 ethylene glycol segments;
the second block comprises 5 to about 100 amino acid units derived from aspartic acid, glutamic acid, or a combination of aspartic acid and glutamic acid; and
two or more side chains of the second block are covalently linked to a therapeutic agent through a linker of the formula: 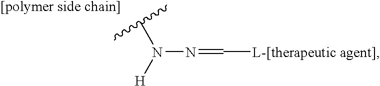
wherein L is a direct bond or a linking group.

13. A polymer comprising formula I: 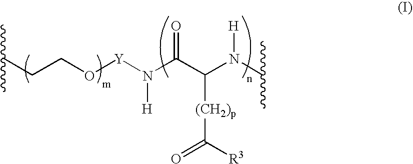
wherein
m is about 10 to about 600;
n is about 10 to about 100;
p is 1, 2, 3, or 4;
Y is a linking group comprising one to twenty carbon atoms, optionally interrupted by one to eight oxygen atoms, nitrogen atoms, or amide groups, and optionally substituted with one to eight oxo groups;
each R³ is independently —OH, a hydroxyl protecting group, an optionally substituted or protected amino group, —NH—NH₂, or —NH—N═C-L-[drug] wherein L is a direct bond or a linking group; and
at least two R³ groups comprise different drugs;
or a salt thereof.

14. The polymer of claim 13 that has formula II: 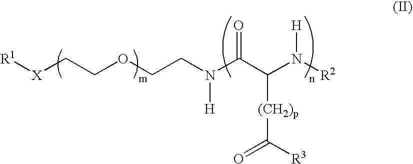
wherein
m is about 10 to about 600;
n is about 10 to about 100;
p is 1, 2, 3, or 4;
R¹ is H, alkyl, or a hydroxyl or nitrogen protecting group;
X is O, NH, or absent;
R² is H or a nitrogen protecting group; and
each R³ is independently OH, a hydroxyl protecting group, —NH—NH₂, or —NH—N═C-L-[drug] where L is a direct bond or a linking group;
or a salt thereof.

15. The polymer of claim 14 wherein the therapeutic agents comprise aclarubicin, apicidin, bortezomib, benzyloxycarbonyl-L-leucyl-L-leucyl-L-leucinal, cyclopamine-KAAD, cucurbitacin, dolastatin, doxorubicin, fenritinide, geldanamycin, herbimycin A, 2-methoxyestradiol, paclitaxel, radicicol, rapamycin, triptolide, wortmannin, or a combination thereof.

16. A micelle comprising a plurality of polymers of claim 1, wherein the therapeutic agents reside toward the core of the micelle and the ethylene glycol segments of the polymers align toward the corona of the micelle.

17. A micelle formulation comprising a plurality of block polymers comprising a first block and a second block;
wherein
the first block comprises two or more ethylene glycol segments;
the second block comprises two or more amino acid units derived from aspartic acid, glutamic acid, or a combination of aspartic acid and glutamic acid;
at least one amino acid side chain of the second block is covalently linked to a therapeutic agent through a hydrazide moiety; and
the micelles of the formulation comprise at least two different therapeutic agents.

18. The micelle formulation of claim 17 wherein each individual micelle of the formulation comprises only one type of therapeutic agent.

19. The micelle formulation of claim 17 wherein each individual micelle comprises two or more therapeutic agent and wherein each individual polymer of each micelle comprises only one type of therapeutic agent.

20. A method of inhibiting the growth of cancer cells or killing cancer cells comprising contacting the cells with an effective amount of the micelle formulation of claim 17.

21. A method of treating cancer comprising administering to a patient in need of cancer treatment a therapeutically effective amount of the micelle formulation of claim 17.

22. The method of claim 21 wherein cancer treatment comprises delivering two or more drugs to a tumor, and wherein the ratio of drug types delivered to the tumor is determined by controlling the ratio of polymers individually comprising different therapeutic agents that are used to prepare the micelles of the micelle formulation.

23. A method of delivering a therapeutic agent to an organ or a tumor comprising administering the micelle formulation of claim 17 to the organ or cell, wherein the polymers of the micelles hydrolyze to release the therapeutic agents upon encountering a pH of less than about 7.
